(19) United States
(12) Patent Application Publication
(10) Pub. No.: US 2022/0364182 A1
(43) Pub. Date: Nov. 17, 2022

(54) METHODS AND COMPOSITIONS FOR DETECTING ecDNA

(71) Applicants: The Regents of the University of California, Oakland, CA (US); Ludwig Institute for Cancer Research Ltd, Zurich (CH)

(72) Inventors: Paul Mischel, La Jolla, CA (US); Vineet Bafna, La Jolla, CA (US); Jens Christian Luebeck, La Jolla, CA (US); Sihan Wu, La Jolla, CA (US)

(21) Appl. No.: 17/746,748

(22) Filed: May 17, 2022

Related U.S. Application Data
(60) Provisional application No. 63/189,462, filed on May 17, 2021.

(51) Int. Cl. C12Q 1/6886

(52) U.S. Cl. CPC C12Q 1/6886

(57) ABSTRACT

Provided herein, inter alia, are methods of detecting the presence of extrachromosomal DNA (ecDNA) in esophageal cells obtained from a subject who has esophageal cancer or who is at risk of developing esophageal cancer. Provided are methods for treating esophageal cancer, including detecting the presence of ecDNA and administering an esophageal cancer treatment.

### CROSS-REFERENCES TO RELATED APPLICATIONS

This application claims the benefit of U.S. Provisional Application No. 63/189,462, filed May 17, 2021, which is incorporated herein by reference in its entirety and for all purposes.

### BACKGROUND

Barrett's esophagus is a premalignant condition characterized by a histological change in the cells lining the esophagus. Barrett's esophagus confers an eleven-fold lifetime increase in esophageal cancer risk, but the annual risk of transformation to cancer is only about 0.12% per year. Consequently, Barrett's esophagus patients are routinely screened for cancer because it is not currently well understood which patients will go on to develop cancer. Endoscopic surveillance of patients with BE is not a risk-free procedure. It is estimated that the risk of development of adenocarcinoma is one in every 200 or 300 patients with BE, whereas the risk of a major complication from an endoscopic procedure is one in 1000 esophagogastroscopies, Identifying the key features that predict transformation from Barrett's esophagus into cancer is essential for patient risk stratification, early cancer detection and treatment.

Provided herein, inter alia, are solutions to these and other problems in the art.

### BRIEF SUMMARY OF THE INVENTION

In an aspect is provided a method for detecting extrachromosomal DNA (ecDNA) in a subject who has esophageal cancer or is at risk of developing esophageal cancer; the method including: i) obtaining a biological sample containing a plurality esophageal cells from the subject; and ii) detecting the presence of an extrachromosomal DNA (ecDNA) in the plurality of esophageal cells.

In an aspect is provided a method for detecting extrachromosomal DNA (ecDNA) in a subject at risk of developing esophageal cancer; the method including detecting the presence of extrachromosomal DNA (ecDNA) in a plurality of esophageal cells obtained from the subject.

In another aspect is provided a method of treating esophageal cancer in a subject in need thereof, the method including: i) obtaining a biological sample containing a plurality esophageal cells from the subject; ii) detecting the presence of extrachromosomal DNA (ecDNA) in the plurality of esophageal cells; and iii) administering an esophageal cancer treatment to the subject.

In another aspect is provided a method of treating esophageal cancer in a subject in need thereof, the method including administering an esophageal cancer treatment to the subject wherein the presence of extrachromosomal DNA (ecDNA) in a plurality of esophageal cells obtained from a biological sample from the subject has been detected. The esophageal cancer treatment can include mucosal resection, esophagectomy, radiation therapy, chemotherapy, chemoradiation therapy, laser therapy, electrocoagulation, immunotherapy, or targeted therapy.

### BRIEF DESCRIPTION OF THE DRAWINGS

FIGS. 1A-1D. show schematics detailing steps of the Fred Hutchinson Cancer Research Center (FHCRC) clinical study described herein. FIG. 1A is a schematic overview of the clinical study design. FIG. 1B is a cartoon showing example regions of the esophagus where biopsies and/or histologies were taking from affected esophageal tissues. FIG. 1C is a schematic showing DNA extraction and sequencing. FIG. 1D is a schematic showing an example structure determination of ecDNA by the Amplicon Architect software program.

FIGS. 2A-2D. are oncoplots illustrating that a significant proportion of subjects who develop esophageal cancer (EAC) from Barrett's esophagus (BE) have ecDNA. FIG. 2A shows detection of ecDNA at times T1 and T2 in subjects with cancer outcome (CO). FIG. 2B shows detection of ecDNA at times T1 and T2 in subjects with no cancer outcome (NCO). FIG. 2C shows the area of esophagus where ecDNA was detected in patients with CO and NCO. FIG. 2D shows detection of various genetic abnormalities (fSCNA, BFB and ecDNA) and their region of detection in patients with CO and NCO.

FIG. 2E. shows results illustrating that ecDNA is linked to high grade dysplasia during cancer progression.

FIG. 2F. shows elevated risk of BE progressing to EAC if ecDNA is present.

FIGS. 2G and 2H. shows, via overlapping amplicon similarity score (FIG. 2G) and probability density (FIG. 2H), that certain regions of DNA are being commonly selected for during progression of BE to EAC.

FIGS. 3A-3C. show schematics and data from the Medical Research Council Cancer Unit (MRCCU) cohort (UK) cross sectional study. FIG. 3A illustrates subsets of patients with EAC, high-grade dysplasia, and BE without HGD or EAC. FIG. 3B illustrates the subsets of patients from FIG. 3A who have the presence of ecDNA. FIG. 3C illustrates that ecDNA is highly correlative with esophageal cancer.

FIGS. 4A-4C. illustrate the study timeline and data obtained from the observational study of a single BE patient over time. FIG. 4A shows a timeline of the progression from BE to EAC in the patient. FIG. 4B shows the overall structure of the circular ecDNA amplicons involved in cancer transformation, and FIG. 4C illustrates detailed structural characteristics of ecDNA in EAC. Results from the study illustrate that there is a central role for ecDNA in driving tumorigenesis.

FIGS. 5A-5D. show the general characteristics of ecDNAs that drive the progression from BE to EAC. FIG. 5A shows the proportion of EAC patients with ecDNA. FIG. 5B shows the size (base pairs) of precancer and cancer ecDNA amplicons. FIG. 5C shows the copy number of precancer and cancer ecDNA. FIG. 5D shows complexity score analysis illustrating the evolution of ecDNA during progression to cancer.

FIG. 5E. shows that ecDNA that drive BE to EAC transition have a high proportion of oncogenes.

FIG. 6A. is a graphic representation of a particular locus which is amplified on ecDNA in two different samples. The conserved region includes the oncogene, indicating that the ecDNAs are used to select and amplify oncogenes to drive cancer formation.

FIGS. 6B and 6C. are results illustrating that ecDNA drives high copy numbers of oncogenes involved BE to EAC transitions, as compared to somatic mutations and copy number changes of other genes. FIG. 6B shows that genes on ecDNA, including oncogenes, achieve a higher copy number when amplified on ecDNA as compared to on chromosomal DNA. FIG. 6C shows oncogenes on ecDNA that are selected for between T1 and T2.

FIGS. 6D and 6E. illustrate that ecDNA includes a wide variety of oncogenes that promote the BE to EAC transition. FIG. 6D shows an example array of different oncogenes that are sometimes present in ecDNA to drive tumor development. FIG. 6E intersects the oncogenes found on ecDNA in the BE to EAC transition in the FHCRC data set (box on the left) with the list of oncogenes amplified on ecDNA in the EAC from the TCGA and PCAWG data sets (bottom box), with the oncogenes amplified in EAC from the TCGA and PCAWG data sets that were not found specifically on ecDNA in those data sets (box on the right).

FIG. 7. shows the odds ratios of various types of genetic alterations, illustrating that there is an elevated risk of BE to EAC progression if ecDNA is detected in a subject.

### DETAILED DESCRIPTION

Provided herein are compositions and methods for detecting extrachromosomal DNA (ecDNA) in a subject at risk of developing cancer. Methods provided include detecting the presence of ecDNA in a plurality of esophageal cells obtained from the subject. The methods provided herein include diagnostic testing for esophageal cancer for a subject in which ecDNA is detected. Further provided are methods for treating a subject for esophageal cancer, wherein ecDNA is detected in esophageal cells obtained from the subject.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

“Nucleic acid” refers to nucleotides (e.g., deoxyribonucleotides or ribonucleotides) and polymers thereof in either single-, double- or multiple-stranded form, or complements thereof, or nucleosides (e.g., deoxyribonucleosides or ribonucleosides). The terms “polynucleotide,” “oligonucleotide,” “oligo” or the like refer, in the usual and customary sense, to a linear sequence of nucleotides. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, tRNA, rRNA, a ribozyme, and guide RNA and any types of DNA, genomic DNA, plasmid DNA, and minicircle DNA, and any fragments thereof. Further examples of polynucleotides include a gene, a gene fragment, an exon, an intron, intergenic DNA (including, without limitation, heterochromatic DNA), cDNA, a recombinant polynucleotide, a branched polynucleotide, a plasmid, a vector, isolated DNA of a sequence, isolated RNA of a sequence, a nucleic acid probe, and a primer. For example, the nucleic acid provided herein can be part of a vector. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that often have various lengths, either deoxyribonucleotides or ribonucleotides, or analogs, derivatives or modifications thereof. Different polynucleotides can have different three-dimensional structures, and sometimes perform various functions, known or unknown.

In embodiments, “nucleic acid” does not include nucleosides. The term “nucleoside” refers, in the usual and customary sense, to a glycosylamine including a nucleobase and a five-carbon sugar (ribose or deoxyribose). Non-limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone are done, in some cases, for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs can be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, in some cases, the term is applied to the polynucleotide molecule itself. This alphabetical representation can be input into databases in a computer having a central processing unit and used for bioinformatics applications such as functional genomics and homology searching. Polynucleotides include, in some cases, one or more non-standard nucleotide(s), nucleotide analog(s) and/or modified nucleotides.

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

The term “extrachromosomal DNA” or “ecDNA” as used herein, refers to a deoxyribonucleotide polymer, that typically includes multiple genes and regulatory regions, that does not form part of a cellular chromosome. In instances, ecDNA includes histone proteins. ecDNA is localized within the nucleus of a cell. ecDNA molecules have a circular structure and are not linear, as compared to cellular chromosomes. In instances, the circular structure of ecDNA allows enhanced chromatin accessibility, altered gene regulation, and amplified transcription. In some aspects, multiple copies of ecDNA carrying an oncogene exist in a single nucleus, and the cell comprising the ecDNA will exhibit increased copy number of the oncogene. If ecDNA includes an oncogene, increased levels of oncogene mRNA can result in tumor growth, progression, or resistance to therapy. The term “ecDNA count” as used herein, refers to the number of ecDNAs determined within a certain volume, e.g., ecDNA count per esophageal cell is the number of ecDNAs measured within a cell.

As used herein, the term “promoter” refers to a sequence of DNA which proteins bind to initiate gene expression. For example, transcription factors may bind a promoter region of a gene to transcribe RNA from DNA.

The terms “operably linked” or “functionally linked”, are interchangeable and denote a physical or functional linkage between two or more elements, e.g., polypeptide sequences or polynucleotide sequences, which permits them to operate in their intended fashion. For example, an operable linkage between a polynucleotide of interest and a regulatory sequence (for example, a promoter is a functional link that allows for expression of the polynucleotide of interest). In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, operably linked elements may be contiguous or non-contiguous.

As used herein, the term “oncogene” refers to a gene capable of transforming a healthy cell into a cancer cell due to mutation or increased expression levels of said gene relative to a healthy cell. An “extrachromosomal oncogene” is an oncogene which forms part of an extrachromosomal DNA molecule. The terms “amplified oncogene” or “oncogene amplification” refer to an oncogene being present at multiple copy numbers (e.g., at least 2 or more) in a cell. Likewise, an “amplified extrachromosomal oncogene” is an oncogene, which is present at multiple copy numbers and the multiple copies of said oncogene form part of one or more extrachromosomal DNA molecules. In embodiments, the oncogene forms part of an extrachromosomal DNA. In embodiments, the amplified oncogene forms part of an extrachromosomal DNA. In embodiments, the extrachromosomal oncogene is EGFR. In embodiments, the extrachromosomal oncogene is MYC. In embodiments, the extrachromosomal oncogene is MYCN. In embodiments, the extrachromosomal oncogene is CCND1. In embodiments, the extrachromosomal oncogene is ERBB2. In embodiments, the extrachromosomal oncogene is CDK4. In embodiments, the extrachromosomal oncogene is CDK6. In embodiments, the extrachromosomal oncogene is BRAF. In embodiments, the extrachromosomal oncogene is MDM2. In embodiments, the extrachromosomal oncogene is MDM4. In embodiments, the oncogene is MDM2. In embodiments, the oncogene is MDM4. In embodiments, the oncogene is CDC6. In embodiments, the oncogene is CSF3. In embodiments, the oncogene is HMGA1. In embodiments, the oncogene is RARA. In embodiments, the oncogene is THRA. In embodiments, the oncogene is KRAS. In embodiments, the oncogene is CTNNB1, GATA6 or TNS4. In embodiments, the oncogene is CTNNB1. In embodiments, the oncogene is GATA6. In embodiments, the oncogene is TNS4.

The word “expression” or “expressed” as used herein in reference to a gene means the transcriptional and/or translational product of that gene. The level of expression of a DNA molecule in a cell is determined, in some cases, on the basis of either the amount of corresponding mRNA that is present within the cell or the amount of protein encoded by that DNA produced by the cell. The level of expression of non-coding nucleic acid molecules (e.g., siRNA), in some cases is detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage is a resistance towards a certain toxin that is presented to the cell, in some cases.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that are taken into account, in some cases, when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “polypeptide,” “peptide” and “protein” are used interchangeably herein to refer to a polymer of amino acid residues, wherein the polymer is, in some cases, conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “isolated”, when applied to a nucleic acid or protein, denotes that the nucleic acid or protein is essentially free of other cellular components with which it is associated in the natural state. It can be, for example, in a homogeneous state and in some cases, is in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window, in some cases, comprises additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which, in some cases, a sequence is compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “EGFR” or “EGFR protein” as provided herein includes any of the recombinant or naturally-occurring forms of epidermal growth factor receptor (EGFR), also known as Proto-oncogene c-ErbB-1, Receptor tyrosine-protein kinase erbB-1, or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR. In embodiments, EGFR is the protein as identified by the UniProt reference number P00533 or a variant or homolog thereto.

The term “c-Myc” or “c-Myc protein” as provided herein includes any of the recombinant or naturally-occurring forms of Myc proto-oncogene protein (c-Myc), also known as Class E basic helix-loop-helix protein 39, bHLHe39, Proto-oncogene c-Myc, Transcription factor p64, or variants or homologs thereof that maintain c-Myc activity (e.g. within at least 50%, 80%, 90%, 95%, 96%, 97%, 98%, 99% or 100% activity compared to c-Myc). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring c-Myc. In embodiments, c-Myc is the protein as identified by UniProt reference number P01106, homolog or functional fragment thereof.

The term “N-Myc” or “N-Myc protein” as provided herein includes any of the recombinant or naturally-occurring forms of the N-myc proto-oncogene protein (N-Myc), also known as Class E basic helix-loop-helix protein 37, bHLHe37, or variants or homologs thereof that maintain N-Myc activity (e.g. within at least 50%, 80%, 90%, 95%, 96%, 97%, 98%, 99% or 100% activity compared to N-Myc). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring N-Myc. In embodiments, N-Myc is the protein as identified by UniProt reference number P04198, homolog or functional fragment thereof.

The term “cyclin D1” or “cyclin D1 protein” as provided herein includes any of the recombinant or naturally-occurring forms of the cyclin D1 protein (cyclin D1), also known as G1/S-specific cyclin-D1, B-cell lymphoma 1 protein, BCL-1, or variants or homologs thereof that maintain cyclin D1 activity (e.g. within at least 50%, 80%, 90%, 95%, 96%, 97%, 98%, 99% or 100% activity compared to cyclin D1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring cyclin D1. In embodiments, cyclin D1 is the protein as identified by UniProt reference number P24385, homolog or functional fragment thereof.

The term “ErbB2” or “ErbB2 protein” as provided herein includes any of the recombinant or naturally-occurring forms of the receptor tyrosine-protein kinase erbB-2 (ErbB2), also known as Metastatic lymph node gene 19 protein, Proto-oncogene c-ErbB-2, Tyrosine kinase-type cell surface receptor HER2, CD340, or variants or homologs thereof that maintain ErbB2 activity (e.g. within at least 50%, 80%, 90%, 95%, 96%, 97%, 98%, 99% or 100% activity compared to ErbB2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ErbB2. In embodiments, ErbB2 is the protein as identified by UniProt reference number P04626, homolog or functional fragment thereof.

The term “CDK4” or “CDK4 protein” as provided herein includes any of the recombinant or naturally-occurring forms of the cyclin dependent kinase 4 (CDK4), also known as Cell division protein kinase 4, PSK-J3, or variants or homologs thereof that maintain CDK4 activity (e.g. within at least 50%, 80%, 90%, 95%, 96%, 97%, 98%, 99% or 100% activity compared to CDK4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CDK4. In embodiments, CDK4 is the protein as identified by UniProt reference number P11802, homolog or functional fragment thereof.

The terms “CDK6” or “CDK6 protein” as provided herein includes any of the recombinant or naturally-occurring forms of the cyclin dependent kinase 6 (CDK6), also known as Cell division protein kinase 6, Serine/threonine-protein kinase PLSTIRE, or variants or homologs thereof that maintain CDK6 activity (e.g. within at least 50%, 80%, 90%, 95%, 96%, 97%, 98%, 99% or 100% activity compared to CDK6). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CDK6. In embodiments, CDK6 is the protein as identified by UniProt reference number Q00534, homolog or functional fragment thereof.

The term “B-Raf” or “B-Raf protein” as provided herein includes any of the recombinant or naturally-occurring forms of the serine/threonine-protein kinase B-Raf (B-Raf), also known as Proto-oncogene B-Raf, p94, v-Raf murine sarcoma viral oncogene homolog B1 or variants or homologs thereof that maintain B-Raf activity (e.g. within at least 50%, 80%, 90%, 95%, 96%, 97%, 98%, 99% or 100% activity compared to B-Raf). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring B-Raf. In embodiments, B-Raf is the protein as identified by UniProt reference number P15056, homolog or functional fragment thereof.

The terms “MDM2” or “MDM2 protein” as provided herein includes any of the recombinant or naturally-occurring forms of E3 ubiquitin-protein ligase Mdm2 (MDM2), also known as Double minute 2 protein, Oncoprotein Mdm2, RING-type E3 ubiquitin transferase Mdm2, p53-binding protein Mdm2, or variants or homologs thereof that maintain MDM2 activity (e.g. within at least 50%, 80%, 90%, 95%, 96%, 97%, 98%, 99% or 100% activity compared to MDM2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MDM2. In embodiments, MDM2 is the protein as identified by UniProt reference number Q00987, homolog or functional fragment thereof.

The term “MDM4” or “MDM4 protein” as provided herein includes any of the recombinant or naturally-occurring forms of MDM4, also known as Double minute 4 protein, Mdm2-like p53-binding protein, Protein Mdmx, p53-binding protein Mdm4, or variants or homologs thereof that maintain MDM4 activity (e.g. within at least 50%, 80%, 90%, 95%, 96%, 97%, 98%, 99% or 100% activity compared to MDM4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MDM4. In embodiments, MDM4 is the protein as identified by UniProt reference number O15151, homolog or functional fragment thereof.

The term “CDC6” or “CDC6 protein” as used herein includes any of the recombinant or naturally-occurring forms of Cell division control protein 6 homolog (CDC6), also known as CDC6-related protein, Cdc18-related protein, or variants or homologs thereof that maintain CDC6 activity (e.g. within at least 50%, 80%, 90%, 95%, 96%, 97%, 98%, 99% or 100% activity compared to CDC6). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CDC6 protein. In embodiments, the CDC6 protein is substantially identical to the protein identified by the UniProt reference number Q99741 or a variant or homolog having substantial identity thereto.

The term “CSF3” or “CSF3 protein” as used herein includes any of the recombinant or naturally-occurring forms of Granulocyte colony-stimulating factor (CSF3), also known as Pluripoietin, or variants or homologs thereof that maintain CSF3 activity (e.g. within at least 50%, 80%, 90%, 95%, 96%, 97%, 98%, 99% or 100% activity compared to CSF3). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CSF3 protein. In embodiments, the CSF3 protein is substantially identical to the protein identified by the UniProt reference number Q99741 or a variant or homolog having substantial identity thereto.

The term “HMGA1” or “HMGA1 protein” as used herein includes any of the recombinant or naturally-occurring forms of High mobility group protein HMG-I/HMG-Y (HMGA1), also known as High mobility group AT-hook protein 1, or variants or homologs thereof that maintain HMGA1 activity (e.g. within at least 50%, 80%, 90%, 95%, 96%, 97%, 98%, 99% or 100% activity compared to HMGA1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring HMGA1 protein. In embodiments, the HMGA1 protein is substantially identical to the protein identified by the UniProt reference number P17096 or a variant or homolog having substantial identity thereto.

The term “RAR-α” or “RAR-α protein” as used herein includes any of the recombinant or naturally-occurring forms of Retinoic acid receptor alpha (RAR-α), also known as RAR-alpha, Nuclear receptor subfamily 1 group B member 1, or variants or homologs thereof that maintain RAR-α activity (e.g. within at least 50%, 80%, 90%, 95%, 96%, 97%, 98%, 99% or 100% activity compared to RAR-α). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring RAR-α protein. In embodiments, the RAR-α protein is substantially identical to the protein identified by the UniProt reference number P10276 or a variant or homolog having substantial identity thereto.

The term “THR-α” or “THR-α protein” as used herein includes any of the recombinant or naturally-occurring forms of Thyroid hormone receptor alpha (THR-α), also known as Nuclear receptor subfamily 1 group A member 1, V-erbA-related protein 7, EAR-7, or variants or homologs thereof that maintain THR-α activity (e.g. within at least 50%, 80%, 90%, 95%, 96%, 97%, 98%, 99% or 100% activity compared to THR-α). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring THR-α protein. In embodiments, the THR-α protein is substantially identical to the protein identified by the UniProt reference number P10827 or a variant or homolog having substantial identity thereto.

The term “K-Ras” or “K-Ras protein” as used herein includes any of the recombinant or naturally-occurring forms of GTPase KRas (K-Ras), or variants or homologs thereof that maintain K-Ras activity (e.g. within at least 50%, 80%, 90%, 95%, 96%, 97%, 98%, 99% or 100% activity compared to K-Ras). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring K-Ras protein. In embodiments, the K-Ras protein is substantially identical to the protein identified by the UniProt reference number P01116 or a variant or homolog having substantial identity thereto.

The term “β-catenin” or “β-catenin protein” as used herein includes any of the recombinant or naturally-occurring forms of Catenin beta-1 (β-catenin), also known as Beta-catenin, or variants or homologs thereof that maintain β-catenin activity (e.g. within at least 50%, 80%, 90%, 95%, 96%, 97%, 98%, 99% or 100% activity compared to β-catenin). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring β-catenin protein. In embodiments, the β-catenin protein is substantially identical to the protein identified by the UniProt reference number P35222 or a variant or homolog having substantial identity thereto.

The term “GATA6” or “GATA6 protein” as used herein includes any of the recombinant or naturally-occurring forms of Transcription factor GATA-6 (GATA6), also known as Beta-catenin, or variants or homologs thereof that maintain GATA6 activity (e.g. within at least 50%, 80%, 90%, 95%, 96%, 97%, 98%, 99% or 100% activity compared to GATA6). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring GATA6 protein. In embodiments, the GATA6 protein is substantially identical to the protein identified by the UniProt reference number Q92908 or a variant or homolog having substantial identity thereto.

The term “Tensin-4” or “Tensin-4 protein” as used herein includes any of the recombinant or naturally-occurring forms of Tensin-4, or variants or homologs thereof that maintain Tensin-4 activity (e.g. within at least 50%, 80%, 90%, 95%, 96%, 97%, 98%, 99% or 100% activity compared to Tensin-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Tensin-4 protein. In embodiments, the Tensin-4 protein is substantially identical to the protein identified by the UniProt reference number Q8IZW8 or a variant or homolog having substantial identity thereto.

The terms “EGFR gene”, “epidermal growth factor receptor gene”, or the like, as used herein refer to the any of the recombinant or naturally-occurring forms of the epidermal growth factor receptor gene or variants or homologs thereof that code for a EGFR capable of maintaining the activity of the EGFR polypeptide (e.g., within at least 50%, 80%, 90%, 95%, 96%, 97%, 98%, 99% or 100% activity compared to EGFR polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring EGFR gene. In embodiments, the EGFR is substantially identical to the nucleic acid sequence corresponding to position 55019017-55211628 of the nucleic acid sequence identified by Accession No. NC_000007.14 or a variant or homolog having substantial identity thereto.

The terms “MYC gene”, “MYC proto-oncogene, bHLH transcription factor gene”, or the like, as used herein refer to the any of the recombinant or naturally-occurring forms of the MYC gene or variants or homologs thereof that code for a MYC capable of maintaining the activity of the MYC polypeptide (e.g., within at least 50%, 80%, 90%, 95%, 96%, 97%, 98%, 99% or 100% activity compared to MYC polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring MYC gene. In embodiments, the MYC is substantially identical to the nucleic acid sequence corresponding to position 127735434-127742951 of the nucleic acid sequence identified by Accession No. NC_000008.11 or a variant or homolog having substantial identity thereto.

The terms “ERBB2 gene”, “erb-b2 receptor tyrosine kinase 2 gene”, or the like, as used herein refer to the any of the recombinant or naturally-occurring forms of the ERBB2 gene or variants or homologs thereof that code for a ERBB2 capable of maintaining the activity of the ERBB2 polypeptide (e.g., within at least 50%, 80%, 90%, 95%, 96%, 97%, 98%, 99% or 100% activity compared to ERBB2 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring ERBB2 gene. In embodiments, the ERBB2 is substantially identical to the nucleic acid sequence corresponding to position 39688094-39728658 of the nucleic acid sequence identified by Accession No. NC_000017.11 or a variant or homolog having substantial identity thereto.

The terms “MYCN gene”, “MYCN proto-oncogene, bHLH transcription factor gene”, or the like, as used herein refer to the any of the recombinant or naturally-occurring forms of the MYCN gene or variants or homologs thereof that code for a MYCN capable of maintaining the activity of the MYCN polypeptide (e.g., within at least 50%, 80%, 90%, 95%, 96%, 97%, 98%, 99% or 100% activity compared to MYCN polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring MYCN gene. In embodiments, the MYCN is substantially identical to the nucleic acid sequence corresponding to position 15940550-15947004 of the nucleic acid sequence identified by Accession No. NC_000002.12 or a variant or homolog having substantial identity thereto.

The terms “KRAS gene”, “KRAS proto-oncogene, GTPase gene”, or the like, as used herein refer to the any of the recombinant or naturally-occurring forms of the KRAS gene or variants or homologs thereof that code for a KRAS capable of maintaining the activity of the KRAS polypeptide (e.g., within at least 50%, 80%, 90%, 95%, 96%, 97%, 98%, 99% or 100% activity compared to KRAS polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring KRAS gene. In embodiments, the KRAS is substantially identical to the nucleic acid sequence corresponding to position 25205246-25250929 of the nucleic acid sequence identified by Accession No. NC_000012.12 or a variant or homolog having substantial identity thereto.

The terms “MDM2 gene”, “MDM2 proto-oncogene gene”, or the like, as used herein refer to the any of the recombinant or naturally-occurring forms of the MDM2 gene or variants or homologs thereof that code for a MDM2 capable of maintaining the activity of the MDM2 polypeptide (e.g., within at least 50%, 80%, 90%, 95%, 96%, 97%, 98%, 99% or 100% activity compared to MDM2 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring MDM2 gene. In embodiments, the MDM2 is substantially identical to the nucleic acid sequence corresponding to position 68808172-68850686 of the nucleic acid sequence identified by Accession No. NC_000012.12 or a variant or homolog having substantial identity thereto.

The terms “MDM4 gene”, “MDM4 proto-oncogene gene”, or the like, as used herein refer to the any of the recombinant or naturally-occurring forms of the MDM4 gene or variants or homologs thereof that code for a MDM4 capable of maintaining the activity of the MDM4 polypeptide (e.g., within at least 50%, 80%, 90%, 95%, 96%, 97%, 98%, 99% or 100% activity compared to MDM4 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring MDM4 gene. In embodiments, the MDM4 is substantially identical to the nucleic acid sequence corresponding to position 204516406-204558120 of the nucleic acid sequence identified by Accession No. NC_000001.11 or a variant or homolog having substantial identity thereto.

The terms “CCND1 gene”, “cyclin D1 gene”, or the like, as used herein refer to the any of the recombinant or naturally-occurring forms of the CCND1 gene or variants or homologs thereof that code for a CCND1 capable of maintaining the activity of the CCND1 polypeptide (e.g., within at least 50%, 80%, 90%, 95%, 96%, 97%, 98%, 99% or 100% activity compared to CCND1 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring CCND1 gene. In embodiments, the CCND1 is substantially identical to the nucleic acid sequence corresponding to position 69641156-69654474 of the nucleic acid sequence identified by Accession No. NC_000011.10 or a variant or homolog having substantial identity thereto.

The terms “CDK4 gene”, “cyclin dependent kinase 4 gene”, or the like, as used herein refer to the any of the recombinant or naturally-occurring forms of the CDK4 gene or variants or homologs thereof that code for a CDK4 capable of maintaining the activity of the CDK4 polypeptide (e.g., within at least 50%, 80%, 90%, 95%, 96%, 97%, 98%, 99% or 100% activity compared to CDK4 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring CDK4 gene. In embodiments, the CDK4 is substantially identical to the nucleic acid sequence corresponding to position 57747727-57753310 of the nucleic acid sequence identified by Accession No. NC_000012.12 or a variant or homolog having substantial identity thereto.

The terms “BRAF gene”, “B-Raf proto-oncogene, serine/threonine kinase gene”, or the like, as used herein refer to the any of the recombinant or naturally-occurring forms of the BRAF gene or variants or homologs thereof that code for a BRAF capable of maintaining the activity of the BRAF polypeptide (e.g., within at least 50%, 80%, 90%, 95%, 96%, 97%, 98%, 99% or 100% activity compared to BRAF polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring BRAF gene. In embodiments, the BRAF is substantially identical to the nucleic acid sequence corresponding to position 140713328-140924929 of the nucleic acid sequence identified by Accession No. NC_000007.14 or a variant or homolog having substantial identity thereto.

The terms “CDC6 gene”, “cell division cycle 6 gene”, or the like, as used herein refer to the any of the recombinant or naturally-occurring forms of the CDC6 gene or variants or homologs thereof that code for a CDC6 capable of maintaining the activity of the CDC6 polypeptide (e.g., within at least 50%, 80%, 90%, 95%, 96%, 97%, 98%, 99% or 100% activity compared to CDC6 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring CDC6 gene. In embodiments, the CDC6 is substantially identical to the nucleic acid sequence corresponding to position 40287879-40304657 of the nucleic acid sequence identified by Accession No. NC_000017.11 or a variant or homolog having substantial identity thereto.

The words “complementary” or “complementarity” refer to the ability of a nucleic acid in a polynucleotide to form a base pair with another nucleic acid in a second polynucleotide. For example, the sequence A-G-T is complementary to the sequence T-C-A. In some cases, complementarity is partial, in which only some of the nucleic acids match according to base pairing, or complete, where all the nucleic acids match according to base pairing.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding occurs, in some cases, by Watson Crick base pairing, Hoogstein binding, or in any other sequence specific manner. In some cases, the complex comprises two strands forming a duplex structure, three or more strands forming a multi stranded complex, a single self-hybridizing strand, or any combination of these. A hybridization reaction, in some cases, constitutes a step in a more extensive process, such as the initiation of PCR, or the cleavage of a polynucleotide by an enzyme. A sequence capable of hybridizing with a given sequence is referred to as the “complement” of the given sequence.

“Contacting” is used in accordance with its plain ordinary meaning and refers to the process of allowing at least two distinct species (e.g. nucleic acids and/or proteins) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting”, in some cases, includes allowing two or more species to react, interact, or physically touch (e.g., bind), wherein in some cases, the two or more species are, for example, an esophageal cancer cell as described herein and a chemotherapeutic as described herein.

As used herein, the terms “binding,” “specific binding” or “specifically binds” refer to two or more molecules forming a complex (e.g., an extrachromosomal nucleic acid protein complex) that is relatively stable under physiologic conditions.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells alternatively include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells are, in some cases, useful when they are naturally nonadherent or have been treated not to adhere to surfaces, for example by trypsinization. “Esophageal cell” as used herein, refers to a cell from the esophagus—the muscular tube connecting the throat (pharynx) with the stomach. The wall of the esophagus from the lumen outwards consists of mucosa, submucosa (connective tissue), layers of muscle fibers between layers of fibrous tissue, and an outer layer of connective tissue. The mucosa is a stratified squamous epithelium of around three layers of squamous cells, which contrasts to the single layer of columnar cells of the stomach. The transition between these two types of epithelium is visible as a zig-zag line. Most of the muscle is smooth muscle although striated muscle predominates in its upper third. It has two muscular rings or sphincters in its wall, one at the top and one at the bottom. The lower sphincter helps to prevent reflux of acidic stomach content. In embodiments, an esophageal cell includes cells from the esophagus that have undergone transformation due to Barrett's Esophagus or esophageal cancer.

“Biological sample” or “sample” refers to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, pleural effusion, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In some embodiments, the sample is obtained from a human. In embodiments, the biological sample includes esophageal epithelial tissue. In embodiments, the biological sample is a sample including esophageal cells.

A “control” or “standard control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. In embodiments, a control is a subject who does not have cancer (e.g. esophageal cancer). In embodiments, a control is a subject who is not at risk of having esophageal cancer. In embodiments, a control is a subject who does not have ecDNA in a plurality of esophageal cells obtained from the subject.

“Patient” or “subject in need thereof” refers to a living organism suffering from or prone to a disease (e.g. esophageal cancer) or condition (e.g. Barrett's esophagus) that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer (e.g. esophageal cancer, lung cancer, ovarian cancer, osteosarcoma, bladder cancer, cervical cancer, liver cancer, kidney cancer, skin cancer (e.g., Merkel cell carcinoma), testicular cancer, leukemia, lymphoma (Mantel cell lymphoma), head and neck cancer, colorectal cancer, prostate cancer, pancreatic cancer, melanoma, breast cancer, neuroblastoma). In embodiments, the cancer is esophageal cancer.

The term “esophageal cancer” refers to a cancer arising from the esophagus. The two main sub-types of esophageal cancer are esophageal squamous-cell carcinoma (ESCC) and esophageal adenocarcinoma (EAC). A number of less common esophageal cancer are also known. Squamous-cell carcinoma arises from the epithelial cells that line the esophagus. Adenocarcinoma arises from glandular cells present in the lower third of the esophagus, often where they have already transformed to an intestinal cell type (a condition known as Barrett's esophagus). Causes of the squamous-cell type include tobacco, alcohol, frequent consumption of hot drinks (e.g. over about 149° F. (65° C.) in temperature), poor diet, and consumption of betel nut. Causes of the adenocarcinoma type include smoking tobacco, obesity, and acid reflux.

The term “carcinoma” refers to a malignant new growth made up of epithelial cells tending to infiltrate the surrounding tissues and give rise to metastases. Exemplary carcinomas that are, in some cases, treated with compositions and/or methods provided herein include, for example, esophageal adenocarcinoma (EAC).

The term “associated” or “associated with” in the context of a substance or substance activity or function associated with a disease (e.g., cancer (e.g. esophageal cancer)) means that the disease (e.g. cancer, (e.g. esophageal cancer)) is caused by (in whole or in part), or a symptom of the disease is caused by (in whole or in part) the substance or substance activity or function.

The term “aberrant” as used herein refers to different from normal. When used to describe enzymatic activity or protein function, aberrant refers to activity or function that is greater or less than a normal control or the average of normal non-diseased control samples. In some cases, aberrant activity refers to an amount of activity that results in a disease, wherein returning the aberrant activity to a normal or non-disease-associated amount (e.g., by administering a compound or using a method as described herein), results in reduction of the disease or one or more disease symptoms.

The term “signaling pathway” as used herein refers to a series of interactions between cellular and optionally extra-cellular components (e.g., proteins, nucleic acids, small molecules, ions, lipids) that conveys a change in one component to one or more other components, which in turn sometimes conveys a change to additional components, which is optionally propagated to other signaling pathway components.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

As used herein the terms “treatment,” “treat,” or “treating” refers to a method of reducing the effects of one or more symptoms of a disease (e.g., cancer) or condition (e.g., Barrett's esophagus). Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

An “effective amount” or “therapeutically effective amount” are used interchangeably and refer to an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and, in some cases, occurs only after administration of a series of doses. Thus, in some cases, a prophylactically effective amount is administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20^{th} Edition, 2003, Gennaro, Ed., Lippincott, Williams & Wilkins). For any composition (e.g. NK cell composition) described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The term “prevent” refers to a decrease in the occurrence of disease symptoms in a patient. As indicated above, in some cases, the prevention is complete (no detectable symptoms) or partial, such that fewer symptoms are observed than would likely occur absent treatment.

Dosages, in some cases, are varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” is used in accordance with its plain and ordinary meaning in the art and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

### Methods of Detection

Provided herein are methods useful for detecting extrachromosomal DNA (ecDNA) in a subject who has esophageal cancer or is at risk of developing esophageal cancer. In embodiments, the subject with esophageal cancer has not been diagnosed with esophageal cancer. In embodiments, the subject with esophageal cancer has early stage (e.g. stage 1) esophageal cancer. A subject “at risk of developing esophageal cancer” refers to a subject that has a condition that, in some cases, progresses to a pathogenic state. For example, the condition includes Barrett's esophagus (BE), gastroesophageal reflux disease (GERD), obesity, achalasia, human papilloma virus (HPV) infection, nutrient deficiencies, chronic use of tobacco, or chronic use of alcohol, or combinations thereof. In embodiments, a subject at risk of developing esophageal cancer is a subject with Barrett's esophagus. In embodiments, a subject at risk of developing esophageal cancer is a subject with gastroesophageal reflux disease. In embodiments, a subject at risk of developing esophageal cancer is a subject with obesity. In embodiments, a subject at risk of developing esophageal cancer is a subject with achalasia. In embodiments, a subject at risk of developing esophageal cancer is a subject with human papilloma virus (HPV) infection. In embodiments, a subject at risk of developing esophageal cancer is a subject with nutrient deficiencies. In embodiments, a subject at risk of developing esophageal cancer is a subject with chronic use of tobacco. In embodiments, a subject at risk of developing esophageal cancer is a subject with chronic use of alcohol. In instances, conditions that progress to a pathogenic state (e.g. BE, GERD, etc.) cause the esophageal squamous epithelial cells which typically line a healthy esophagus to undergo metaplasia or dysplasia. For example, the esophageal cells that typically line the esophagus are, in some cases, replaced by columnar epithelial cells typically found in the intestinal epithelium. The esophageal cells are sometimes replaced by parietal cells and chief cells typically found in the stomach. In an aspect is provided a method for detecting extrachromosomal DNA (ecDNA) in a subject who has is esophageal cancer or who is at risk of developing esophageal cancer, the method including detecting the presence of extrachromosomal DNA (ecDNA) in a plurality of esophageal cells obtained from the subject. In an aspect is provided a method for detecting extrachromosomal DNA (ecDNA) in a subject who has esophageal cancer or who is at risk of developing esophageal cancer, the method including i) obtaining a biological sample containing a plurality esophageal cells from said subject; and ii) detecting the presence of an extrachromosomal DNA (ecDNA) in the plurality of esophageal cells.

In embodiments, the subject has Barrett's esophagus (BE). The terms “Barrett's esophagus” or “BE” refers to a condition in which cells typically found in a subject's esophagus (e.g. squamous epithelial cells) are replaced with cells (e.g. columnar epithelial cells) that are not normally found in the esophagus. Typically, columnar epithelial cells, cells characterized by a height longer than the width of the cell, replace squamous epithelial cells normally found in the esophagus. Columnar epithelial cells are sometimes characterized by nuclei positioned near the base of the cell. Columnar epithelial cells in some cases include cells typically found in the intestine (e.g. colon, duodenum) or stomach. Columnar epithelial cells in some cases resemble cells typically found in the intestine (e.g. colon, duodenum) or stomach. In instances, goblet cells replace squamous epithelial cells typically found in the esophagus. Goblet cells are sometimes characterized by a narrow base and rounded, expanded apical portion. Typically, the nuclei of goblet cells are found at the base of the cell. In contrast, squamous epithelial cells are typically flat and have centrally-located nuclei. BE is sometimes caused by conditions including GERD. For example, prolonged exposure to acid due to acid reflux sometimes causes prolonged injury to the squamous epithelium of the esophagus, thereby causing cells to undergo metaplasia. In instances, BE can lead to esophageal cancer. For example, changes in esophageal tissue (e.g., dysplasia), including replacement of squamous epithelial cells with abnormal (e.g. immature cells, not fully differentiated cells) cells in subjects with BE can lead to adenocarcinoma. Thus, in some cases, a subject who has BE is a subject at risk of developing esophageal cancer.

For the methods provided herein, in embodiments, the biological sample includes esophageal tissue. In embodiments, the biological sample includes esophageal tissue from the distal esophagus (e.g. the region directly adjacent to the stomach). In embodiments, the biological sample includes esophageal epithelial tissue. In embodiments, the biological sample includes esophageal epithelial tissue from the distal esophagus.

In embodiments, the plurality of epithelial cells have metaplasia or dysplasia. In embodiments, the plurality of epithelial cells have metaplasia. In embodiments, the plurality of epithelial cells have dysplasia. “Metaplasia” is used in accordance with its plain ordinary meaning and refers to replacement of a cell normally found in a biological sample or tissue with a cell not typically found in the sample or tissue. In metaplasia, the cells replacing the original cells are typically mature or fully differentiated. For example, in some cases, a biological sample including esophageal cells has metaplasia if the biological sample includes columnar epithelial cells resembling those typically found in the intestines. Metaplasia is typically caused by an abnormal stimulus, for example, excess stomach acid caused by acid reflux (e.g. caused by GERD). Metaplasia sometimes occurs due to inability of normal cells to survive an abnormal stimulus. Typically, metaplasia will stop if the abnormal stimulus stops.

“Dysplasia” is used in accordance with its plain ordinary meaning and refers to replacement of cells normally found within a biological sample or tissue with cells not normally found within a biological sample or tissue, wherein the cells not normally found are immature or are not fully differentiated. In dysplasia, the growth and differentiation of cells therefore is sometimes decreased. In instances, the number of immature or not fully differentiated cells increases, while the number of mature cells decreases. Dysplasia is associated with pathogeny (e.g. high grade dysplasia, etc.) in some cases, due to an irregularity that prevents cell maturation or differentiation within a particular tissue (e.g. esophagus). In the case of Barrett's esophagus, in embodiments, a five-tiered system is used when evaluating dysplasia in Barrett's metaplastic epithelium: negative for dysplasia, indefinite for dysplasia, low-grade dysplasia, high-grade dysplasia, and intramucosal adenocarcinoma. In instances, dysplasia transforms to neoplasia, wherein growth of cells not typically found in the biological sample or tissue is uncontrolled. Neoplasia is sometimes characterized by loss of complete cell differentiation. For instance, in some cases of Barrett's esophagus, dysplasia in the esophageal epithelium transforms to neoplasia.

In embodiments, the plurality of esophageal cells have high grade dysplasia. High grade dysplasia refers to the presence of abnormal cells found in a biological sample or tissue. For example, the cells that replace the squamous epithelial cells in the esophagus sometimes have abnormal shape or lack of nuclear polarity (e.g. the nuclei of cells have an inconsistent relationship to each other). For example, in some cases, the abnormal cells lack shape, size or nuclear uniformity with squamous epithelial cells. For example, the abnormal cells sometimes lack shape, size or nuclear uniformity with columnar epithelial cells or goblet cells. High-grade dysplasia can be characterized by distortion of glandular architecture.

As described above and throughout the specification, the plurality of esophageal cells sometimes have metaplasia or dysplasia and therefore, in some cases, include other types of cells not typically found in the biological sample (e.g. esophageal tissue). In embodiments, the plurality of esophageal cells include columnar epithelial cells. In embodiments, the plurality of esophageal cells include goblet cells. For example, in some cases, a subject at risk for esophageal cancer has a condition (e.g. Barrett's esophagus, GERD, etc.) in which columnar epithelial cells or goblet cells replace squamous epithelial cells typically found in the esophagus. Columnar epithelial cells are characterized by their elongated, column shape and typically have a height of at least about four types their width. Columnar epithelial cells are typically found in the lining of the intestines, including the colon, or stomach. Goblet cells are typically found in the intestines and have the capability to secrete mucin. The subject at risk for esophageal cancer, in some cases, has a condition in which squamous epithelial cells typically found in the esophagus are replaced by columnar epithelial cells. In some cases, the subject at risk for esophageal cancer has a condition in which squamous epithelial cells typically found in the esophagus are replaced by cells typically found in the stomach and or intestines. In embodiments, the plurality of esophageal cells include columnar epithelial cells, chief cells, parietal cells, enterocytes, Paneth cells, or enteroendocrine cells, or combinations thereof. Chief cell and parietal cell are typically found in the epithelium of the stomach. Enterocytes are a type of columnar epithelial cells that typically line the inner surface of the small and large intestines. Paneth cells are cells in the small intestine epithelium and on intestinal glands. Enteroendorine cells are found in the gastrointestinal tract and pancreas. In embodiments, the plurality of esophageal cells include chief cells. In embodiments, the plurality of esophageal cells include parietal cells. In embodiments, the plurality of esophageal cells include enterocytes. In embodiments, the plurality of esophageal cells include Paneth cells. In embodiments, the plurality of esophageal cells include enteroendocrine cells.

In embodiments, the plurality of esophageal cells include immature cells or cells that are not fully differentiated. The plurality of esophageal cells therefore, in some cases, includes immature cells of chief cell lineage, parietal cell lineage, enterocyte lineage, Paneth cell lineage, or enteroendocrine cell lineage. Therefore, in embodiments, the plurality of esophageal cells include characteristics of chief cells, parietal cells, enterocytes, Paneth cells, enteroendocrine cells, or combinations thereof. A characteristic of a cell often includes cell shape, for example a columnar cell shape (e.g. columnar epithelial cells, chief cells, enteroendocrine cells) or pyramidal cell shape (e.g. paneth cells, parietal cells). A characteristic of a cell is, in some cases, secretion of mucin. A characteristic of a cell can be position of the nucleus (e.g. basal nucleus position). In embodiments, the plurality of esophageal cells include characteristics of chief cells. In embodiments, the plurality of esophageal cells include characteristics of parietal cells. In embodiments, the plurality of esophageal cells include characteristics of enterocytes. In embodiments, the plurality of esophageal cells include characteristics of Paneth cells. In embodiments, the plurality of esophageal cells include characteristics of enteroendocrine cells.

In embodiments, the plurality of esophageal cells include characteristics of columnar epithelial cells, mucus cells, colon cells, stomach cells or cells of the duodenum. In embodiments, the plurality of esophageal cells include a characteristic of columnar epithelial cells. In embodiments, the plurality of esophageal cells include a characteristic of mucus cells. In embodiments, the plurality of esophageal cells include a characteristic of colon cells. In embodiments, the plurality of esophageal cells include a characteristic of stomach cells. In embodiments, the plurality of esophageal cells include a characteristic of cells of the duodenum.

In embodiments, the method further includes determining the ecDNA count per esophageal cell. Methods for determining the number of ecDNAs within a cell are known in the art, and include for example, ecDETECT. eDETECT includes use of a fluorescent dye to quantify ecDNA in metaphase cells. For example, in metaphase, chromosomal structures are separated from ecDNA and ecDNA is, in some cases, accurately detected by staining cells with a fluorescent DNA-binding dye (e.g. DAPI) ecDETECT is described in detail in U.S. Patent Publication No. 2018/0355416A1, which is incorporated by reference herein in its entirety and for all purposes. In embodiments, the average ecDNA count per cell from about 1 to about 20. In embodiments, the average ecDNA count per cell from about 2 to about 20. In embodiments, the average ecDNA count per cell from about 3 to about 20. In embodiments, the average ecDNA count per cell from about 4 to about 20. In embodiments, the average ecDNA count per cell from about 5 to about 20. In embodiments, the average ecDNA count per cell from about 6 to about 20. In embodiments, the average ecDNA count per cell from about 7 to about 20. In embodiments, the average ecDNA count per cell from about 8 to about 20. In embodiments, the average ecDNA count per cell from about 9 to about 20. In embodiments, the average ecDNA count per cell from about 10 to about 20. In embodiments, the average ecDNA count per cell from about 11 to about 20. In embodiments, the average ecDNA count per cell from about 12 to about 20. In embodiments, the average ecDNA count per cell from about 13 to about 20. In embodiments, the average ecDNA count per cell from about 14 to about 20. In embodiments, the average ecDNA count per cell from about 15 to about 20. In embodiments, the average ecDNA count per cell from about 16 to about 20. In embodiments, the average ecDNA count per cell from about 17 to about 20. In embodiments, the average ecDNA count per cell from about 18 to about 20.

In embodiments, the average ecDNA count per cell from about 1 to about 19. In embodiments, the average ecDNA count per cell from about 1 to about 18. In embodiments, the average ecDNA count per cell from about 1 to about 17. In embodiments, the average ecDNA count per cell from about 1 to about 16. In embodiments, the average ecDNA count per cell from about 1 to about 15. In embodiments, the average ecDNA count per cell from about 1 to about 14. In embodiments, the average ecDNA count per cell from about 1 to about 13. In embodiments, the average ecDNA count per cell from about 1 to about 12. In embodiments, the average ecDNA count per cell from about 1 to about 11. In embodiments, the average ecDNA count per cell from about 1 to about 10. In embodiments, the average ecDNA count per cell from about 1 to about 9. In embodiments, the average ecDNA count per cell from about 1 to about 8. In embodiments, the average ecDNA count per cell from about 1 to about 7. In embodiments, the average ecDNA count per cell from about 1 to about 6. In embodiments, the average ecDNA count per cell from about 1 to about 5. In embodiments, the average ecDNA count per cell from about 1 to about 4. In embodiments, the average ecDNA count per cell from about 1 to about 3. In embodiments, the average ecDNA count per cell about 1, about 2, about 3, about 4, about 5, about 6, about 7, about 8, about 9, about 10, about 11, about 12, about 13, about 14, about 15, about 16, about 17, about 18, about 19, or about 20.

In embodiments, the average ecDNA count per cell from about 1 to about 40. In embodiments, the average ecDNA count per cell from about 4 to about 40. In embodiments, the average ecDNA count per cell from about 8 to about 40. In embodiments, the average ecDNA count per cell from about 12 to about 40. In embodiments, the average ecDNA count per cell from about 16 to about 40. In embodiments, the average ecDNA count per cell from about 1 to about 20. In embodiments, the average ecDNA count per cell from about 24 to about 40. In embodiments, the average ecDNA count per cell from about 28 to about 40. In embodiments, the average ecDNA count per cell from about 32 to about 40. In embodiments, the average ecDNA count per cell from about 36 to about 40.

In embodiments, the average ecDNA count per cell from about 1 to about 36. In embodiments, the average ecDNA count per cell from about 1 to about 32. In embodiments, the average ecDNA count per cell from about 1 to about 28. In embodiments, the average ecDNA count per cell from about 1 to about 24. In embodiments, the average ecDNA count per cell from about 1 to about 20. In embodiments, the average ecDNA count per cell from about 1 to about 16. In embodiments, the average ecDNA count per cell from about 1 to about 12. In embodiments, the average ecDNA count per cell from about 1 to about 8. In embodiments, the average ecDNA count per cell from about 1 to about 4. In embodiments, the average ecDNA count per cell about 1, 4, 8, 12, 16, 20, 24, 28, 32, 36, or 40.

In embodiments, the average ecDNA count per esophageal cell is from about 1 to about 100. In embodiments, the average ecDNA count per esophageal cell is from about 10 to about 100. In embodiments, the average ecDNA count per esophageal cell is from about 20 to about 100. In embodiments, the average ecDNA count per esophageal cell is from about 30 to about 100. In embodiments, the average ecDNA count per esophageal cell is from about 40 to about 100. In embodiments, the average ecDNA count per esophageal cell is from about 50 to about 100. In embodiments, the average ecDNA count per esophageal cell is from about 60 to about 100. In embodiments, the average ecDNA count per esophageal cell is from about 70 to about 100. In embodiments, the average ecDNA count per esophageal cell is from about 80 to about 100. In embodiments, the average ecDNA count per esophageal cell is from about 90 to about 100.

In embodiments, the average ecDNA count per esophageal cell is from about 1 to about 90. In embodiments, the average ecDNA count per esophageal cell is from about 1 to about 80. In embodiments, the average ecDNA count per esophageal cell is from about 1 to about 70. In embodiments, the average ecDNA count per esophageal cell is from about 1 to about 60. In embodiments, the average ecDNA count per esophageal cell is from about 1 to about 50. In embodiments, the average ecDNA count per esophageal cell is from about 1 to about 40. In embodiments, the average ecDNA count per esophageal cell is from about 1 to about 30. In embodiments, the average ecDNA count per esophageal cell is from about 1 to about 20. In embodiments, the average ecDNA count per esophageal cell is about 1, 10, 20, 30, 40, 50, 60, 70, 80, 90, or 100.

In embodiments, the average ecDNA count per esophageal cell is at least 1, at least 2, at least 3, at least 4, at least 5, at least 6, at least 7, at least 8, at least 9, or at least 10. In embodiments, the ecDNA count per esophageal cell is at least 1. In embodiments, the ecDNA count per esophageal cell is at least 2. In embodiments, the average ecDNA count per esophageal cell is at least 3. In embodiments, the average ecDNA count per esophageal cell is at least 4. In embodiments, the average ecDNA count per esophageal cell is at least 5. In embodiments, the average ecDNA count per esophageal cell is at least 6. In embodiments, the average ecDNA count per esophageal cell is at least 7. In embodiments, the average ecDNA count per esophageal cell is at least 8. In embodiments, the average ecDNA count per esophageal cell is at least 9. In embodiments, the average ecDNA count per esophageal cell is at least 10. In embodiments, the average ecDNA count per esophageal cell is about 1. In embodiments, the average ecDNA count per esophageal cell is about 2. In embodiments, the average ecDNA count per esophageal cell is about 3. In embodiments, t the average ecDNA count per esophageal cell is about 4. In embodiments, the average ecDNA count per esophageal cell is about 5. In embodiments, the average ecDNA count per esophageal cell is about 6. In embodiments, the average ecDNA count per esophageal cell is about 7. In embodiments, the average ecDNA count per esophageal cell is about 8. In embodiments, the average ecDNA count per esophageal cell is about 9. In embodiments, the average ecDNA count per esophageal cell is about 10.

In embodiments, the average ecDNA count per esophageal cell is at least 15, at least 20, at least 25, at least 30, at least 35, or at least 40. In embodiments, the average ecDNA count per esophageal cell is at least 15. In embodiments, the average ecDNA count per esophageal cell is at least 20. In embodiments, the average ecDNA count per esophageal cell is at least 25. In embodiments, the average ecDNA count per esophageal cell is at least 30. In embodiments, the average ecDNA count per esophageal cell is at least 35. In embodiments, the average ecDNA count per esophageal cell is at least 40. In embodiments, the average ecDNA count per esophageal cell is about 15. In embodiments, the average ecDNA count per esophageal cell is 20. In embodiments, the average ecDNA count per esophageal cell is about 25. In embodiments, the average ecDNA count per esophageal cell is about 30. In embodiments, the average ecDNA count per esophageal cell is about 35. In embodiments, the average ecDNA count per esophageal cell is about 40.

In embodiments, the average ecDNA count per esophageal cell is at least 50, at least 60, at least 70, at least 80, at least 90, or at least 100. In embodiments, the average ecDNA count per esophageal cell is at least 50. In embodiments, the average ecDNA count per esophageal cell is at least 60. In embodiments, the average ecDNA count per esophageal cell is at least 70. In embodiments, the average ecDNA count per esophageal cell is at least 80. In embodiments, the average ecDNA count per esophageal cell is at least 90. In embodiments, the average ecDNA count per esophageal cell is at least 100. In embodiments, the average ecDNA count per esophageal cell is about 50. In embodiments, the average ecDNA count per esophageal cell is about 60. In embodiments, the average ecDNA count per esophageal cell is about 70. In embodiments, the average ecDNA count per esophageal cell is about 80. In embodiments, the average ecDNA count per esophageal cell is about 90. In embodiments, the average ecDNA count per esophageal cell is about 100.

In embodiments, the detecting includes detection of an amplified oncogene in the plurality of esophageal cells. In embodiments, the detecting includes determining the average copy number of an amplified oncogene in the plurality of esophageal cells. In embodiments, the oncogene is EGFR, MYC, MYCN, CCND1, ERBB2, CDK4, CDK6, BRAF, MDM2, MDM4, CDC6, CSF3, HMGA1, RARA, THRA, KRAS, CTNNB1, GATA6 or TNS4. In embodiments, the oncogene is EGFR. In embodiments, the oncogene is MYC. In embodiments, the oncogene is MYCN. In embodiments, the oncogene is CCND1. In embodiments, the oncogene is ERBB2. In embodiments, the oncogene is CDK4. In embodiments, the oncogene is CDK6. In embodiments, the oncogene is BRAF. In embodiments, the oncogene is MDM2. In embodiments, the oncogene is MDM4. In embodiments, the oncogene is CDC6. In embodiments, the oncogene is CSF3. In embodiments, the oncogene is HMGA1. In embodiments, the oncogene is RARA. In embodiments, the oncogene is THRA. In embodiments, the oncogene is KRAS. In embodiments, the oncogene is CTNNB1, GATA6 or TNS4. In embodiments, the oncogene is GATA6. In embodiments, the oncogene is TNS4.

In embodiments, the average copy number of the amplified oncogene in the plurality of esophageal cells is from about 2 to about 32. In embodiments, the average copy number of the amplified oncogene in the plurality of esophageal cells is from about 4 to about 32. In embodiments, the average copy number of the amplified oncogene in the plurality of esophageal cells is from about 6 to about 32. In embodiments, the average copy number of the amplified oncogene in the plurality of esophageal cells is from about 8 to about 32. In embodiments, the average copy number of the amplified oncogene in the plurality of esophageal cells is from about 10 to about 32. In embodiments, the average copy number of the amplified oncogene in the plurality of esophageal cells is from about 12 to about 32. In embodiments, the average copy number of the amplified oncogene in the plurality of esophageal cells is from about 14 to about 32. In embodiments, the average copy number of the amplified oncogene in the plurality of esophageal cells is from about 16 to about 32. In embodiments, the average copy number of the amplified oncogene in the plurality of esophageal cells is from about 18 to about 32. In embodiments, the average copy number of the amplified oncogene in the plurality of esophageal cells is from about 20 to about 32. In embodiments, the average copy number of the amplified oncogene in the plurality of esophageal cells is from about 22 to about 32. In embodiments, the average copy number of the amplified oncogene in the plurality of esophageal cells is from about 24 to about 32. In embodiments, the average copy number of the amplified oncogene in the plurality of esophageal cells is from about 26 to about 32. In embodiments, the average copy number of the amplified oncogene in the plurality of esophageal cells is from about 28 to about 32. In embodiments, the average copy number of the amplified oncogene in the plurality of esophageal cells is from about 30 to about 32.

In embodiments, the average copy number of the amplified oncogene in the plurality of esophageal cells is from about 2 to about 30. In embodiments, the average copy number of the amplified oncogene in the plurality of esophageal cells is from about 2 to about 28. In embodiments, the average copy number of the amplified oncogene in the plurality of esophageal cells is from about 2 to about 26. In embodiments, the average copy number of the amplified oncogene in the plurality of esophageal cells is from about 2 to about 24. In embodiments, the average copy number of the amplified oncogene in the plurality of esophageal cells is from about 2 to about 22. In embodiments, the average copy number of the amplified oncogene in the plurality of esophageal cells is from about 2 to about 20. In embodiments, the average copy number of the amplified oncogene in the plurality of esophageal cells is from about 2 to about 18. In embodiments, the average copy number of the amplified oncogene in the plurality of esophageal cells is from about 2 to about 16. In embodiments, the average copy number of the amplified oncogene in the plurality of esophageal cells is from about 2 to about 14. In embodiments, the average copy number of the amplified oncogene in the plurality of esophageal cells is from about 2 to about 12. In embodiments, the average copy number of the amplified oncogene in the plurality of esophageal cells is from about 2 to about 10. In embodiments, the average copy number of the amplified oncogene in the plurality of esophageal cells is from about 2 to about 8. In embodiments, the average copy number of the amplified oncogene in the plurality of esophageal cells is from about 2 to about 6. In embodiments, the average copy number of the amplified oncogene in the plurality of esophageal cells is from about 2 to about 4. In embodiments, the average copy number of the amplified oncogene in the plurality of esophageal cells is about 2, 4, 6, 8, 10, 12, 14, 16, 18, 20, 22, 24, 26, 28, 30, or 32.

In embodiments, the average copy number of the amplified oncogene in the plurality of esophageal cells is from about 2 to about 140. In embodiments, the average copy number of the amplified oncogene in the plurality of esophageal cells is from about 10 to about 140. In embodiments, the average copy number of the amplified oncogene in the plurality of esophageal cells is from about 20 to about 140. In embodiments, the average copy number of the amplified oncogene in the plurality of esophageal cells is from about 30 to about 140. In embodiments, the average copy number of the amplified oncogene in the plurality of esophageal cells is from about 40 to about 140. In embodiments, the average copy number of the amplified oncogene in the plurality of esophageal cells is from about 50 to about 140. In embodiments, the average copy number of the amplified oncogene in the plurality of esophageal cells is from about 60 to about 140. In embodiments, the average copy number of the amplified oncogene in the plurality of esophageal cells is from about 70 to about 140. In embodiments, the average copy number of the amplified oncogene in the plurality of esophageal cells is from about 80 to about 140. In embodiments, the average copy number of the amplified oncogene in the plurality of esophageal cells is from about 90 to about 140. In embodiments, the average copy number of the amplified oncogene in the plurality of esophageal cells is from about 100 to about 140. In embodiments, the average copy number of the amplified oncogene in the plurality of esophageal cells is from about 110 to about 140. In embodiments, the average copy number of the amplified oncogene in the plurality of esophageal cells is from about 120 to about 140. In embodiments, the average copy number of the amplified oncogene in the plurality of esophageal cells is from about 130 to about 140.

In embodiments, the average copy number of the amplified oncogene in the plurality of esophageal cells is from about 2 to about 130. In embodiments, the average copy number of the amplified oncogene in the plurality of esophageal cells is from about 2 to about 120. In embodiments, the average copy number of the amplified oncogene in the plurality of esophageal cells is from about 2 to about 110. In embodiments, the average copy number of the amplified oncogene in the plurality of esophageal cells is from about 2 to about 100. In embodiments, the average copy number of the amplified oncogene in the plurality of esophageal cells is from about 2 to about 90. In embodiments, the average copy number of the amplified oncogene in the plurality of esophageal cells is from about 2 to about 80. In embodiments, the average copy number of the amplified oncogene in the plurality of esophageal cells is from about 2 to about 70. In embodiments, the average copy number of the amplified oncogene in the plurality of esophageal cells is from about 2 to about 60. In embodiments, the average copy number of the amplified oncogene in the plurality of esophageal cells is from about 2 to about 130. In embodiments, the average copy number of the amplified oncogene in the plurality of esophageal cells is from about 2 to about 130. In embodiments, the average copy number of the amplified oncogene in the plurality of esophageal cells is from about 2 to about 130. In embodiments, the average copy number of the amplified oncogene in the plurality of esophageal cells is from about 2 to about 130. In embodiments, the average copy number of the amplified oncogene in the plurality of esophageal cells is from about 2 to about 130. In embodiments, the average copy number of the amplified oncogene in the plurality of esophageal cells is about 2, 10, 20, 30, 40, 50, 60, 70, 80, 90, 100, 110, 120, 130, or 140.

In embodiments, the average copy number of the amplified oncogene in the plurality of esophageal cells is at least 2, 4, 6, 8, 10, 12, 14, 16, 18, 20, 22, 24, 26, 28, 30, or 32. In embodiments, the average copy number of the amplified oncogene in the plurality of esophageal cells is at least 2. In embodiments, the average copy number of the amplified oncogene in the plurality of esophageal cells is at least 4. In embodiments, the average copy number of the amplified oncogene in the plurality of esophageal cells is at least 6. In embodiments, the average copy number of the amplified oncogene in the plurality of esophageal cells is at least 8. In embodiments, the average copy number of the amplified oncogene in the plurality of esophageal cells is at least 10. In embodiments, the average copy number of the amplified oncogene in the plurality of esophageal cells is at least 12. In embodiments, the average copy number of the amplified oncogene in the plurality of esophageal cells is at least 14. In embodiments, the average copy number of the amplified oncogene in the plurality of esophageal cells is at least 16. In embodiments, the average copy number of the amplified oncogene in the plurality of esophageal cells is at least 18. In embodiments, the average copy number of the amplified oncogene in the plurality of esophageal cells is at least 20. In embodiments, the average copy number of the amplified oncogene in the plurality of esophageal cells is at least 22. In embodiments, the average copy number of the amplified oncogene in the plurality of esophageal cells is at least 24. In embodiments, the average copy number of the amplified oncogene in the plurality of esophageal cells is at least 26. In embodiments, the average copy number of the amplified oncogene in the plurality of esophageal cells is at least 28. In embodiments, the average copy number of the amplified oncogene in the plurality of esophageal cells is at least 30. In embodiments, the average copy number of the amplified oncogene in the plurality of esophageal cells is at least 32. In embodiments, the average copy number of the amplified oncogene in the plurality of esophageal cells is about 2. In embodiments, the average copy number of the amplified oncogene in the plurality of esophageal cells is about 4. In embodiments, the average copy number of the amplified oncogene in the plurality of esophageal cells is about 6. In embodiments, the average copy number of the amplified oncogene in the plurality of esophageal cells is about 8. In embodiments, the average copy number of the amplified oncogene in the plurality of esophageal cells is about 10. In embodiments, the average copy number of the amplified oncogene in the plurality of esophageal cells is about 12. In embodiments, the average copy number of the amplified oncogene in the plurality of esophageal cells is about 14. In embodiments, the average copy number of the amplified oncogene in the plurality of esophageal cells is about 16. In embodiments, the average copy number of the amplified oncogene in the plurality of esophageal cells is about 18. In embodiments, the average copy number of the amplified oncogene in the plurality of esophageal cells is about 20. In embodiments, the average copy number of the amplified oncogene in the plurality of esophageal cells is about 22. In embodiments, the average copy number of the amplified oncogene in the plurality of esophageal cells is about 24. In embodiments, the average copy number of the amplified oncogene in the plurality of esophageal cells is about 26. In embodiments, the average copy number of the amplified oncogene in the plurality of esophageal cells is about 28. In embodiments, the average copy number of the amplified oncogene in the plurality of esophageal cells is about 30. In embodiments, the average copy number of the amplified oncogene in the plurality of esophageal cells is about 32.

In embodiments, the average copy number of the amplified oncogene in the plurality of esophageal cells is at least 10, 20, 30, 40, 50, 60, 70, 80, 90, 100, 110, 120, 130, or 140. In embodiments, the average copy number of the amplified oncogene in the plurality of esophageal cells is at least 10. In embodiments, the average copy number of the amplified oncogene in the plurality of esophageal cells is at least 20. In embodiments, the average copy number of the amplified oncogene in the plurality of esophageal cells is at least 30. In embodiments, the average copy number of the amplified oncogene in the plurality of esophageal cells is at least 40. In embodiments, the average copy number of the amplified oncogene in the plurality of esophageal cells is at least 50. In embodiments, the average copy number of the amplified oncogene in the plurality of esophageal cells is at least 60. In embodiments, the average copy number of the amplified oncogene in the plurality of esophageal cells is at least 70. In embodiments, the average copy number of the amplified oncogene in the plurality of esophageal cells is at least 80. In embodiments, the average copy number of the amplified oncogene in the plurality of esophageal cells is at least 90. In embodiments, the average copy number of the amplified oncogene in the plurality of esophageal cells is at least 100. In embodiments, the average copy number of the amplified oncogene in the plurality of esophageal cells is at least 110. In embodiments, the average copy number of the amplified oncogene in the plurality of esophageal cells is at least 120. In embodiments, the average copy number of the amplified oncogene in the plurality of esophageal cells is at least 130. In embodiments, the average copy number of the amplified oncogene in the plurality of esophageal cells is at least 140. In embodiments, the average copy number of the amplified oncogene in the plurality of esophageal cells is about 10. In embodiments, the average copy number of the amplified oncogene in the plurality of esophageal cells is about 20. In embodiments, the average copy number of the amplified oncogene in the plurality of esophageal cells is about 30. In embodiments, the average copy number of the amplified oncogene in the plurality of esophageal cells is about 40. In embodiments, the average copy number of the amplified oncogene in the plurality of esophageal cells is about 50. In embodiments, the average copy number of the amplified oncogene in the plurality of esophageal cells is about 60. In embodiments, the average copy number of the amplified oncogene in the plurality of esophageal cells is about 70. In embodiments, the average copy number of the amplified oncogene in the plurality of esophageal cells is about 80. In embodiments, the average copy number of the amplified oncogene in the plurality of esophageal cells is about 90. In embodiments, the average copy number of the amplified oncogene in the plurality of esophageal cells is about 100. In embodiments, the average copy number of the amplified oncogene in the plurality of esophageal cells is about 110. In embodiments, the average copy number of the amplified oncogene in the plurality of esophageal cells is about 120. In embodiments, the average copy number of the amplified oncogene in the plurality of esophageal cells is about 130. In embodiments, the average copy number of the amplified oncogene in the plurality of esophageal cells is about 140.

For the methods provided herein, in some cases, the ecDNA includes one or more oncogenes. Applicants have found that genes, including oncogenes, achieve a higher copy number when amplified on ecDNA relative to chromosomal DNA. The presence of oncogenes therefore may contribute to transition of dysplasia to neoplasia in esophageal cancer. Thus, in embodiments, the ecDNA includes a sequence encoding an oncogene. In embodiments, the ecDNA sequence includes a DNA oncogene sequence. In embodiments, the method further includes determining the presence of an oncogene on the ecDNA. In embodiments, the oncogene is EGFR, MYC, MYCN, CCND1, ERBB2, CDK4, CDK6, BRAF, MDM2, MDM4, CDC6, CSF3, HMGA1, RARA, THRA, KRAS, CTNNB1, GATA6 or TNS4. In embodiments, the oncogene is EGFR. In embodiments, the oncogene is MYC. In embodiments, the oncogene is MYCN. In embodiments, the oncogene is CCND1. In embodiments, the oncogene is ERBB2. In embodiments, the oncogene is CDK4. In embodiments, the oncogene is CDK6. In embodiments, the oncogene is BRAF. In embodiments, the oncogene is MDM2. In embodiments, the oncogene is MDM4. In embodiments, the oncogene is CDC6. In embodiments, the oncogene is CSF3. In embodiments, the oncogene is HMGA1. In embodiments, the oncogene is RARA. In embodiments, the oncogene is THRA. In embodiments, the oncogene is KRAS. In embodiments, the oncogene is CTNNB1, GATA6 or TNS4. In embodiments, the oncogene is GATA6. In embodiments, the oncogene is TNS4.

In embodiments, the average copy number of the oncogene encoded on the ecDNA is from about 2 to about 40. In embodiments, the average copy number of the oncogene encoded on the ecDNA is from about 4 to about 40. In embodiments, the average copy number of the oncogene encoded on the ecDNA is from about 6 to about 40. In embodiments, the average copy number of the oncogene encoded on the ecDNA is from about 8 to about 40. In embodiments, the average copy number of the oncogene encoded on the ecDNA is from about 10 to about 40. In embodiments, the average copy number of the oncogene encoded on the ecDNA is from about 12 to about 40. In embodiments, the average copy number of the oncogene encoded on the ecDNA is from about 14 to about 40. In embodiments, the average copy number of the oncogene encoded on the ecDNA is from about 16 to about 40. In embodiments, the average copy number of the oncogene encoded on the ecDNA is from about 18 to about 40. In embodiments, the average copy number of the oncogene encoded on the ecDNA is from about 20 to about 40. In embodiments, the average copy number of the oncogene encoded on the ecDNA is from about 22 to about 40. In embodiments, the average copy number of the oncogene encoded on the ecDNA is from about 24 to about 40. In embodiments, the average copy number of the oncogene encoded on the ecDNA is from about 26 to about 40. In embodiments, the average copy number of the oncogene encoded on the ecDNA is from about 28 to about 40. In embodiments, the average copy number of the oncogene encoded on the ecDNA is from about 30 to about 40. In embodiments, the average copy number of the oncogene encoded on the ecDNA is from about 32 to about 40. In embodiments, the average copy number of the oncogene encoded on the ecDNA is from about 34 to about 40. In embodiments, the average copy number of the oncogene encoded on the ecDNA is from about 36 to about 40. In embodiments, the average copy number of the oncogene encoded on the ecDNA is from about 38 to about 40.

In embodiments, the average copy number of the oncogene encoded on the ecDNA is from about 2 to about 38. In embodiments, the average copy number of the oncogene encoded on the ecDNA is from about 2 to about 36. In embodiments, the average copy number of the oncogene encoded on the ecDNA is from about 2 to about 34. In embodiments, the average copy number of the oncogene encoded on the ecDNA is from about 2 to about 32. In embodiments, the average copy number of the oncogene encoded on the ecDNA is from about 2 to about 30. In embodiments, the average copy number of the oncogene encoded on the ecDNA is from about 2 to about 28. In embodiments, the average copy number of the oncogene encoded on the ecDNA is from about 2 to about 26. In embodiments, the average copy number of the oncogene encoded on the ecDNA is from about 2 to about 24. In embodiments, the average copy number of the oncogene encoded on the ecDNA is from about 2 to about 22. In embodiments, the average copy number of the oncogene encoded on the ecDNA is from about 2 to about 20. In embodiments, the average copy number of the oncogene encoded on the ecDNA is from about 2 to about 18. In embodiments, the average copy number of the oncogene encoded on the ecDNA is from about 2 to about 16. In embodiments, the average copy number of the oncogene encoded on the ecDNA is from about 2 to about 14. In embodiments, the average copy number of the oncogene encoded on the ecDNA is from about 2 to about 12. In embodiments, the average copy number of the oncogene encoded on the ecDNA is from about 2 to about 10. In embodiments, the average copy number of the oncogene encoded on the ecDNA is from about 2 to about 8. In embodiments, the average copy number of the oncogene encoded on the ecDNA is from about 2 to about 6. In embodiments, the average copy number of the oncogene encoded on the ecDNA is from about 2 to about 4. In embodiments, the average copy number of the oncogene encoded on the ecDNA is about 2, 4, 6, 8, 10, 12, 14, 16, 18, 20, 22, 24, 26, 28, 30, 32, 34, 36, or 38.

In embodiments, the average copy number of the oncogene encoded on the ecDNA is from about 10 to about 130. In embodiments, the average copy number of the oncogene encoded on the ecDNA is from about 20 to about 130. In embodiments, the average copy number of the oncogene encoded on the ecDNA is from about 30 to about 130. In embodiments, the average copy number of the oncogene encoded on the ecDNA is from about 40 to about 130. In embodiments, the average copy number of the oncogene encoded on the ecDNA is from about 50 to about 130. In embodiments, the average copy number of the oncogene encoded on the ecDNA is from about 60 to about 130. In embodiments, the average copy number of the oncogene encoded on the ecDNA is from about 70 to about 130. In embodiments, the average copy number of the oncogene encoded on the ecDNA is from about 80 to about 130. In embodiments, the average copy number of the oncogene encoded on the ecDNA is from about 90 to about 130. In embodiments, the average copy number of the oncogene encoded on the ecDNA is from about 100 to about 130. In embodiments, the average copy number of the oncogene encoded on the ecDNA is from about 110 to about 130. In embodiments, the average copy number of the oncogene encoded on the ecDNA is from about 120 to about 130.

In embodiments, the average copy number of the oncogene encoded on the ecDNA is from about 10 to about 120. In embodiments, the average copy number of the oncogene encoded on the ecDNA is from about 10 to about 110. In embodiments, the average copy number of the oncogene encoded on the ecDNA is from about 10 to about 100. In embodiments, the average copy number of the oncogene encoded on the ecDNA is from about 10 to about 90. In embodiments, the average copy number of the oncogene encoded on the ecDNA is from about 10 to about 80. In embodiments, the average copy number of the oncogene encoded on the ecDNA is from about 10 to about 70. In embodiments, the average copy number of the oncogene encoded on the ecDNA is from about 10 to about 60. In embodiments, the average copy number of the oncogene encoded on the ecDNA is from about 10 to about 50. In embodiments, the average copy number of the oncogene encoded on the ecDNA is from about 10 to about 40. In embodiments, the average copy number of the oncogene encoded on the ecDNA is from about 10 to about 30. In embodiments, the average copy number of the oncogene encoded on the ecDNA is from about 10 to about 20. In embodiments, the average copy number of the oncogene encoded on the ecDNA is about 10, 20, 30, 40, 50, 60, 70, 80, 90, 100, 110, 120, or 130.

In embodiments, the average copy number of the oncogene encoded on the ecDNA is at least 2, 4, 6, 8, 10, 12, 14, 16, 18, 20, 22, 24, 26, 28, 30, 32, 34, 36, or 38. In embodiments, the average copy number of the oncogene encoded on the ecDNA is at least 2. In embodiments, the average copy number of the oncogene encoded on the ecDNA is at least 4. In embodiments, the average copy number of the oncogene encoded on the ecDNA is at least 6. In embodiments, the average copy number of the oncogene encoded on the ecDNA is at least 8. In embodiments, the average copy number of the oncogene encoded on the ecDNA is at least 10. In embodiments, the average copy number of the oncogene encoded on the ecDNA is at least 12. In embodiments, the average copy number of the oncogene encoded on the ecDNA is at least 14. In embodiments, the average copy number of the oncogene encoded on the ecDNA is at least 16. In embodiments, the average copy number of the oncogene encoded on the ecDNA is at least 18. In embodiments, the average copy number of the oncogene encoded on the ecDNA is at least 20. In embodiments, the average copy number of the oncogene encoded on the ecDNA is at least 22. In embodiments, the average copy number of the oncogene encoded on the ecDNA is at least 24. In embodiments, the average copy number of the oncogene encoded on the ecDNA is at least 26. In embodiments, the average copy number of the oncogene encoded on the ecDNA is at least 28. In embodiments, the average copy number of the oncogene encoded on the ecDNA is at least 30. In embodiments, the average copy number of the oncogene encoded on the ecDNA is at least 32. In embodiments, the average copy number of the oncogene encoded on the ecDNA is at least 34. In embodiments, the average copy number of the oncogene encoded on the ecDNA is at least 36. In embodiments, the average copy number of the oncogene encoded on the ecDNA is at least 38. In embodiments, the average copy number of the oncogene encoded on the ecDNA is about 2. In embodiments, the average copy number of the oncogene encoded on the ecDNA is about 4. In embodiments, the average copy number of the oncogene encoded on the ecDNA is about 6. In embodiments, the average copy number of the oncogene encoded on the ecDNA is about 8. In embodiments, the average copy number of the oncogene encoded on the ecDNA is about 10. In embodiments, the average copy number of the oncogene encoded on the ecDNA is about 12. In embodiments, the average copy number of the oncogene encoded on the ecDNA is about 14. In embodiments, the average copy number of the oncogene encoded on the ecDNA is about 16. In embodiments, the average copy number of the oncogene encoded on the ecDNA is about 18. In embodiments, the average copy number of the oncogene encoded on the ecDNA is about 20. In embodiments, the average copy number of the oncogene encoded on the ecDNA is about 22. In embodiments, the average copy number of the oncogene encoded on the ecDNA is about 24. In embodiments, the average copy number of the oncogene encoded on the ecDNA is about 26. In embodiments, the average copy number of the oncogene encoded on the ecDNA is about 28. In embodiments, the average copy number of the oncogene encoded on the ecDNA is about 30. In embodiments, the average copy number of the oncogene encoded on the ecDNA is about 32. In embodiments, the average copy number of the oncogene encoded on the ecDNA is about 34. In embodiments, the average copy number of the oncogene encoded on the ecDNA is about 36. In embodiments, the average copy number of the oncogene encoded on the ecDNA is about 38.

In embodiments, the average copy number of the oncogene encoded on the ecDNA is at least 40, 50, 60, 70, 80, 90, 100, 110, 120, or 130. In embodiments, the average copy number of the oncogene encoded on the ecDNA is at least 40. In embodiments, the average copy number of the oncogene encoded on the ecDNA is at least 50. In embodiments, the average copy number of the oncogene encoded on the ecDNA is at least 60. In embodiments, the average copy number of the oncogene encoded on the ecDNA is at least 70. In embodiments, the average copy number of the oncogene encoded on the ecDNA is at least 80. In embodiments, the average copy number of the oncogene encoded on the ecDNA is at least 90. In embodiments, the average copy number of the oncogene encoded on the ecDNA is at least 100. In embodiments, the average copy number of the oncogene encoded on the ecDNA is at least 110. In embodiments, the average copy number of the oncogene encoded on the ecDNA is at least 120. In embodiments, the average copy number of the oncogene encoded on the ecDNA is at least 130. In embodiments, the average copy number of the oncogene encoded on the ecDNA is about 40. In embodiments, the average copy number of the oncogene encoded on the ecDNA is about 50. In embodiments, the average copy number of the oncogene encoded on the ecDNA is about 60. In embodiments, the average copy number of the oncogene encoded on the ecDNA is about 70. In embodiments, the average copy number of the oncogene encoded on the ecDNA is about 80. In embodiments, the average copy number of the oncogene encoded on the ecDNA is about 90. In embodiments, the average copy number of the oncogene encoded on the ecDNA is about 100. In embodiments, the average copy number of the oncogene encoded on the ecDNA is about 110. In embodiments, the average copy number of the oncogene encoded on the ecDNA is about 120. In embodiments, the average copy number of the oncogene encoded on the ecDNA is about 130.

For the method provided herein, in embodiments, detecting the presence of an ecDNA includes optical microscopy, electron microscopy, density gradient centrifugation, assay of transposase-accessible chromatin with visualization (ATAC-see), fluorescence in situ hybridization (FISH), third-generation sequencing (TGS), circle-sequencing (Circle-Seq), chromatin immunoprecipitation sequencing (ChIP-seq), circular chromosome conformation capture combined with high-throughput sequencing (4C-seq), proximity ligation-assisted ChIP-seq (PLAC-seq), assay for targeting accessible chromatin with high-throughput sequencing (ATAC-seq), or combinations thereof. For example, in some cases, ecDNA is visualized using DNA dyes and optical microscopy, or with ultrahigh-resolution microscope technology (e.g. 3D-SIM). In other examples, transposase-mediated imaging technology used in ATAC-see is useful for imaging, cell sorting and deep sequencing of accessible genomes for revealing the identity of imaged DNA. In other examples, fluorescently labelled DNA probes (e.g. FISH) is sometimes used to detect known ecDNA. In other examples, circular DNA is purified from cells using a circular DNA column, and remaining linear DNA is digested, a DNA polymerase is used to amplification of circular DNA, and high-throughput sequencing can be used to detect ecDNA (e.g. circle-SEQ). In other examples, in some cases, histone modifications specific to ecDNA are exploited for ChiP, and DNA fragments enriched via ChIP are subject to NGS (ChIP-seq). In embodiments, detecting ecDNA is used using ecDETECT as described above.

For the method provided herein, in embodiments, the subject is at an elevated risk of esophageal cancer if the presence of ecDNA is detected in the esophageal cells, relative to a subject wherein the presence of ecDNA is not detected in said esophageal cells. In embodiments, the subject has Barrett's esophagus (BE), gastroesophageal reflux disease (GERD), obesity, achalasia, human papilloma virus (HPV) infection, nutrient deficiencies, chronic use of tobacco, or chronic use of alcohol, or combinations thereof. For example, in some cases, a subject with ecDNA in the plurality of esophageal cells has at least 0.05×, 0.1×, 0.2×, 0.3×, 0.5×, 0.6×, 0.7×, 0.8×, 0.9×, 1×, 1.5×, 2×, 2.5×, 3×, 3.5×, 4×, 4.5×, 5×, 6×, 7×, 8×, 9×, 10×, 20×, 30×, 40×, 50×, 60×, 70×, 80×, 90×, or 100× increased probability of developing esophageal cancer relative to a subject who does not have ecDNA in the plurality of esophageal cells.

In embodiments, a subject with ecDNA in the plurality of esophageal cells has at least 0.05× increased probability of developing esophageal cancer relative to a subject who does not have ecDNA in the plurality of esophageal cells. In embodiments, a subject with ecDNA in the plurality of esophageal cells has at least 0.1× increased probability of developing esophageal cancer relative to a subject who does not have ecDNA in the plurality of esophageal cells. In embodiments, a subject with ecDNA in the plurality of esophageal cells has at least 0.2× increased probability of developing esophageal cancer relative to a subject who does not have ecDNA in the plurality of esophageal cells. In embodiments, a subject with ecDNA in the plurality of esophageal cells has at least 0.3× increased probability of developing esophageal cancer relative to a subject who does not have ecDNA in the plurality of esophageal cells. In embodiments, a subject with ecDNA in the plurality of esophageal cells has at least 0.4× increased probability of developing esophageal cancer relative to a subject who does not have ecDNA in the plurality of esophageal cells. In embodiments, a subject with ecDNA in the plurality of esophageal cells has at least 0.5× increased probability of developing esophageal cancer relative to a subject who does not have ecDNA in the plurality of esophageal cells. In embodiments, a subject with ecDNA in the plurality of esophageal cells has at least 0.6× increased probability of developing esophageal cancer relative to a subject who does not have ecDNA in the plurality of esophageal cells. In embodiments, a subject with ecDNA in the plurality of esophageal cells has at least 0.7× increased probability of developing esophageal cancer relative to a subject who does not have ecDNA in the plurality of esophageal cells. In embodiments, a subject with ecDNA in the plurality of esophageal cells has at least 0.8× increased probability of developing esophageal cancer relative to a subject who does not have ecDNA in the plurality of esophageal cells. In embodiments, a subject with ecDNA in the plurality of esophageal cells has at least 0.9× increased probability of developing esophageal cancer relative to a subject who does not have ecDNA in the plurality of esophageal cells. In embodiments, a subject with ecDNA in the plurality of esophageal cells has at least 1× increased probability of developing esophageal cancer relative to a subject who does not have ecDNA in the plurality of esophageal cells. In embodiments, a subject with ecDNA in the plurality of esophageal cells has at least 1.5× increased probability of developing esophageal cancer relative to a subject who does not have ecDNA in the plurality of esophageal cells. In embodiments, a subject with ecDNA in the plurality of esophageal cells has at least 2× increased probability of developing esophageal cancer relative to a subject who does not have ecDNA in the plurality of esophageal cells. In embodiments, a subject with ecDNA in the plurality of esophageal cells has at least 2.5× increased probability of developing esophageal cancer relative to a subject who does not have ecDNA in the plurality of esophageal cells. In embodiments, a subject with ecDNA in the plurality of esophageal cells has at least 3× increased probability of developing esophageal cancer relative to a subject who does not have ecDNA in the plurality of esophageal cells. In embodiments, a subject with ecDNA in the plurality of esophageal cells has at least 3.5× increased probability of developing esophageal cancer relative to a subject who does not have ecDNA in the plurality of esophageal cells. In embodiments, a subject with ecDNA in the plurality of esophageal cells has at least 4× increased probability of developing esophageal cancer relative to a subject who does not have ecDNA in the plurality of esophageal cells. In embodiments, a subject with ecDNA in the plurality of esophageal cells has at least 4.5× increased probability of developing esophageal cancer relative to a subject who does not have ecDNA in the plurality of esophageal cells. In embodiments, a subject with ecDNA in the plurality of esophageal cells has at least 5× increased probability of developing esophageal cancer relative to a subject who does not have ecDNA in the plurality of esophageal cells. In embodiments, a subject with ecDNA in the plurality of esophageal cells has at least 6× increased probability of developing esophageal cancer relative to a subject who does not have ecDNA in the plurality of esophageal cells. In embodiments, a subject with ecDNA in the plurality of esophageal cells has at least 7× increased probability of developing esophageal cancer relative to a subject who does not have ecDNA in the plurality of esophageal cells. In embodiments, a subject with ecDNA in the plurality of esophageal cells has at least 8× increased probability of developing esophageal cancer relative to a subject who does not have ecDNA in the plurality of esophageal cells. In embodiments, a subject with ecDNA in the plurality of esophageal cells has at least 9× increased probability of developing esophageal cancer relative to a subject who does not have ecDNA in the plurality of esophageal cells. In embodiments, a subject with ecDNA in the plurality of esophageal cells has at least 10× increased probability of developing esophageal cancer relative to a subject who does not have ecDNA in the plurality of esophageal cells. In embodiments, a subject with ecDNA in the plurality of esophageal cells has at least 20× increased probability of developing esophageal cancer relative to a subject who does not have ecDNA in the plurality of esophageal cells. In embodiments, a subject with ecDNA in the plurality of esophageal cells has at least 30× increased probability of developing esophageal cancer relative to a subject who does not have ecDNA in the plurality of esophageal cells. In embodiments, a subject with ecDNA in the plurality of esophageal cells has at least 40× increased probability of developing esophageal cancer relative to a subject who does not have ecDNA in the plurality of esophageal cells. In embodiments, a subject with ecDNA in the plurality of esophageal cells has at least 50× increased probability of developing esophageal cancer relative to a subject who does not have ecDNA in the plurality of esophageal cells. In embodiments, a subject with ecDNA in the plurality of esophageal cells has at least 60× increased probability of developing esophageal cancer relative to a subject who does not have ecDNA in the plurality of esophageal cells. In embodiments, a subject with ecDNA in the plurality of esophageal cells has at least 70× increased probability of developing esophageal cancer relative to a subject who does not have ecDNA in the plurality of esophageal cells. In embodiments, a subject with ecDNA in the plurality of esophageal cells has at least 80× increased probability of developing esophageal cancer relative to a subject who does not have ecDNA in the plurality of esophageal cells. In embodiments, a subject with ecDNA in the plurality of esophageal cells has at least 90× increased probability of developing esophageal cancer relative to a subject who does not have ecDNA in the plurality of esophageal cells. In embodiments, a subject with ecDNA in the plurality of esophageal cells has at least 100× increased probability of developing esophageal cancer relative to a subject who does not have ecDNA in the plurality of esophageal cells.

In embodiments, the method further includes administering to the subject an esophageal cancer-preventative treatment. In embodiments, the method further includes administering to the subject an esophageal cancer-preventative treatment, wherein the presence of ecDNA is detected in the plurality of esophageal cells. An esophageal cancer-preventative treatment refers to a treatment that includes removal of tissue from the esophagus, wherein the tissue contains cells including ecDNA, cells having metaplasia, cells having dysplasia, or a combination thereof. In embodiments, an esophageal cancer preventative treatment includes removal of tissue from the esophagus, wherein the tissue contains cells including ecDNA. In embodiments, an esophageal cancer preventative treatment includes removal of tissue from the esophagus, wherein the tissue includes cells having dysplasia and wherein ecDNA was detected in the cells. In embodiments, an esophageal cancer preventative treatment includes removal of tissue from the esophagus, wherein the tissue includes cells having metaplasia and wherein ecDNA was detected in the cells. In embodiments, the esophageal cancer-preventative treatment is endoscopic resection, cryotherapy, radiofrequency ablation, or surgery. In embodiments, the esophageal cancer-preventative treatment is endoscopic resection. Endoscopic resection refers to a procedure for removing abnormal tissue from the esophagus. In instances, an electrical snare (a wire fed through the end of an endoscope) tightens around the abnormal tissue or tumor in the esophagus. An electrical current is, in some cases, passed through the wire, separating the tissue or tumor from the rest of the esophagus. In embodiments, the esophageal cancer-preventative treatment is cryotherapy. Cryotherapy refers to exposure of abnormal tissue or a tumor within the esophagus to cold temperatures, typically by contacting the tissue or tumor with liquid nitrogen via an endoscope. In embodiments, the esophageal cancer-preventative treatment is radiofrequency ablation. Radiofrequency ablation refers to use of radio frequency to exposure the abnormal tissue or tumor to heat to slough the tissue or tumor off the rest of the esophagus. In embodiments, the esophageal cancer-preventative treatment is surgery. For example, in some cases, the surgery includes removal of a section of the esophagus that includes cells having dysplasia.

In embodiments, diagnostic test is performed to detect abnormal tissue or cancer cells if the presence of ecDNA is detected in the plurality of esophageal cells. Thus, in embodiments, the method further includes performing a diagnostic test for esophageal cancer, wherein the presence of ecDNA is detected in the plurality of esophageal cells. In embodiments, the method further includes performing a diagnostic test for esophageal cancer. Diagnostic tests for esophageal cancer are well known in the art and include imaging methods (e.g., x-rays, magnetic fields, sound waves, or detection of radioactive substances, etc.), barium swallow test, computed tomography (CT) scan, CT-guided needle biopsy, magnetic resonance imaging (MRI) scan, positron emission tomography (PET) scan, endoscopy (e.g., upper endoscopy), endoscopy ultrasound, bronchoscopy, thoracoscopy and laparoscopy, and biopsy.

In embodiments, the diagnostic test is barium swallow test, computed tomography (CT) scan, CT-guided needle biopsy, magnetic resonance imaging (MRI) scan, positron emission tomography (PET) scan, endoscopy, endoscopy ultrasound, bronchoscopy, thoracoscopy and laparoscopy, or biopsy. In embodiments, the diagnostic test is barium swallow test. For example, in some cases, a barium swallow test includes administering a liquid including barium to the subject. The barium liquid coats the inner surface of the esophagus and can be imaged, thereby allowing detection of abnormal tissue in the esophagus or pathogenic growths in the esophagus. In embodiments, the diagnostic test is computed tomography (CT) scan. In embodiments, the diagnostic test is CT-guided needle biopsy. For example, in a CT scan, multiple X-rays allow for generation of cross-section images of the esophagus to detecting abnormal tissue or pathogenic growths. In some cases, a biopsy is taken as guided by CT images. In embodiments, the diagnostic test is magnetic resonance imaging (MRI) scan. In embodiments, the diagnostic test is positron emission tomography (PET) scan. For example, in some cases, in a PET scan, a radioactive substance (e.g. FDG) that selectively enters cancer cells is administered to the subject, thereby allowing detection of cancer cells. In embodiments, the diagnostic test is endoscopy. In embodiments, the diagnostic test is endoscopy ultrasound. In embodiments, the diagnostic test is bronchoscopy. In embodiments, the diagnostic test is thoracoscopy. In embodiments, the diagnostic test is laparoscopy. For example, in some cases, an instrument (e.g. endoscope) is inserted into the esophagus, allowing detection of abnormalities and pathogenic cells. In some cases, the instrument is inserted into the esophagus via the mouth (e.g. endoscopy), or via an incision made near the ribs (e.g. thorascopy). In embodiments, the diagnostic test is biopsy. For example a biopsy can detect high grade dysplasia or neoplasia in the cells. For example, a biopsy can be used to obtain esophageal cells for detection of one or more amplified oncogenes (e.g. HER2). For example, a biopsy can be used to obtain cells for detection of gene modifications indicative of esophageal cancer, including microsatellite instabilities or loss of function in mismatch repair genes (e.g., MLH1, MSH2, MSH6 or PMS2).

Applicant has found that the presence of ecDNA in esophageal cells correlates with the incidence of esophageal cancer. Thus, in embodiments, the method further includes treating the subject for esophageal cancer. In embodiments, the method further includes treating the subject for esophageal cancer if ecDNA is detected in the plurality of esophageal cells. In embodiments, the method further includes treating the subject for esophageal cancer, wherein the presence of ecDNA is detected in the plurality of esophageal cells. For example, in some cases, treating the subject for esophageal cancer includes mucosal resection, esophagectomy, radiation therapy, chemotherapy, chemoradiation therapy, laser therapy, electrocoagulation, targeted therapy, or a combination thereof. In embodiments, treating the subject includes mucosal resection. Mucosal resections include removal of abnormal tissue or tumors in the esophagus. The mucosal resection is sometimes performed via an endoscope thereby bypassing the need for surgery. In embodiments, treating the subject includes esophagectomy. Esophagectomy refers to a surgical procedure to remove some or all of the esophagus and then reconstruct the portion of the esophagus that was removed. Typically, reconstruction is completed by use of a portion of another organ (e.g. stomach). In embodiments, treating the subject includes radiation therapy. Radiation therapy refers to use of high-energy rays or particles to kill cancer cells. Radiation therapy, in some cases, is accompanied by chemotherapy or surgery (e.g. esophagectomy). In regards to esophageal cancer, in some cases. external-beam radiation therapy (EBRT) or internal radiation therapy (brachytherapy) is used. In embodiments, treating the subject includes chemotherapy. In regards to esophageal cancer, in some cases, the chemotherapy includes carboplatin and paclitaxel (Taxol), oxaliplatin and either 5-FU or capecitabine, cisplatin and either 5-fluorouracil (5-FU) or capecitabine, cisplatin and irinotecan (Camptosar), or paclitaxel (Taxol) and either 5-FU or capecitabine. In embodiments, the chemotherapy is administered with radiation therapy. In embodiments, treating the subject includes chemoradiation therapy. Chemoradiation therapy refers to the combination of chemotherapy and radiation therapy. In embodiments, treating the subject includes laser therapy. Laser therapy includes use of a high-intensity light source to burn abnormal tissue or tumors. In embodiments, treating the subject includes electrocoagulation. Electrocoagulation refers to a treatment method wherein a probe is passed into the esophagus via an endoscope to burn off abnormal tissue or tumor with an electric current. In embodiments, treating the subject includes targeted therapy. Targeted therapy refers to use of anti-cancer therapeutics that target proteins overexpressed on or specifically expressed by cancer cells. For example, in some cases, for esophageal cancer, targeted therapy includes monoclonal antibodies that target EGFR (cetuximab), VEGF (bevacizumab), HER-2 (trastuzumab), or combinations thereof. In embodiments, treating the subject includes immunotherapy. In some cases, the immunotherapy includes checkpoint inhibitors. In some cases, the immunotherapy includes pembrolizumab or nivolumab.

In embodiments, treating includes administering chemotherapy. In embodiments, the chemotherapy is carboplatin and paclitaxel (Taxol). In embodiments, the chemotherapy is oxaliplatin and 5-FU. In embodiments, the chemotherapy is oxaliplatin and capecitabine. In embodiments, the chemotherapy is cisplatin and 5-fluorouracil (5-FU). In embodiments, the chemotherapy is cisplatin and capecitabine. In embodiments, the chemotherapy is cisplatin and irinotecan (Camptosar). In embodiments, the chemotherapy is paclitaxel (Taxol) and 5-FU. In embodiments, the chemotherapy is paclitaxel (Taxol) and capecitabine. In embodiments, treating includes administering targeted therapy. In embodiments, the targeted therapy is cetuximab, bevacizumab, trastuzumab, or combinations thereof. In embodiments, the targeted therapy is cetuximab. In embodiments, the targeted therapy is bevacizumab. In embodiments, the targeted therapy is trastuzumab. In embodiments, treating includes administering immunotherapy. In some cases, the immunotherapy includes checkpoint inhibitors. In some cases, the immunotherapy includes pembrolizumab or nivolumab. In embodiments, the immunotherapy is pembrolizumab. In embodiments, the immunotherapy is nivolumab.

As described herein, the presence of ecDNA in esophageal cells is correlative with esophageal cancer. Thus, when ecDNA is not detected in the plurality of esophageal cells, in some cases, the subject is monitored for appearance of ecDNA or other indications of esophageal cancer without administration of an esophageal cancer treatment. In embodiments, the method further includes placing the subject on a watch and wait course. In embodiments, the method further includes placing the subject on a watch and wait course, wherein ecDNA is not detected in the plurality of esophageal cells. As used herein, the term “watch and wait” refers to monitoring a subject for the presence of ecDNA or another indication of esophageal cancer without administration of a esophageal cancer treatment, until presence of ecDNA or another indication of esophageal cancer is detected. In embodiments, the monitoring a subject includes performing a diagnostic test for esophageal cancer. In some cases, the diagnostic test is a barium swallow test, computed tomography (CT) scan, CT-guided needle biopsy, magnetic resonance imaging (MRI) scan, positron emission tomography (PET) scan, endoscopy, endoscopy ultrasound, bronchoscopy, thoracoscopy and laparoscopy, or biopsy, as described herein. In embodiments, the monitoring includes performing a diagnostic test every 6 months, once a year, every 2 years, every 3 years, every 4 years, or every 5 years. Thus, in embodiments, monitoring includes performing a diagnostic test on the subject every 6 months. In embodiments, monitoring includes performing a diagnostic test on the subject once a year. In embodiments, monitoring includes performing a diagnostic test on the subject every 2 years. In embodiments, monitoring includes performing a diagnostic test on the subject every 3 years. In embodiments, monitoring includes performing a diagnostic test on the subject every 4 years. In embodiments, monitoring includes performing a diagnostic test on the subject every 5 years. In embodiments, monitoring a subject includes detecting the presence of ecDNA in a biological sample containing a plurality of esophageal cells from the subject, as described herein. In embodiments, the biological sample is obtained by biopsy, wherein the biopsy is performed every 6 months, once a year, every 2 years, every 3 years, every 4 years, or every 5 years. In embodiments, the biopsy is performed every 6 months. In embodiments, the biopsy is performed once a year. In embodiments, the biopsy is performed every 2 years. In embodiments, the biopsy is performed every 3 years. In embodiments, the biopsy is performed every 4 years. In embodiments, the biopsy is performed every 5 years.

In embodiments, the monitoring a subject includes detecting symptoms of esophageal cancer, including difficulty swallowing, chest pain, weight loss, vomiting, bone pain, bleeding into the esophagus, or combinations thereof. In embodiments, the symptom of esophageal cancer is difficulty swallowing, chest pain, weight loss, vomiting, bone pain, bleeding into the esophagus. In embodiments, the symptom of esophageal cancer is difficulty swallowing. In embodiments, the symptom of esophageal cancer is chest pain. In embodiments, the symptom of esophageal cancer is weight loss. In embodiments, the symptom of esophageal cancer is vomiting. In embodiments, the symptom of esophageal cancer is bone pain. In embodiments, the symptom of esophageal cancer is bleeding into the esophagus.

In embodiments, the indication of esophageal cancer is presence of ecDNA in a plurality of esophageal cells in a biological sample obtained from the subject. In embodiments, the indication of esophageal cancer is a symptom of esophageal cancer as described above (e.g. difficulty swallowing, chest pain, weight loss, vomiting, bone pain, bleeding into the esophagus, or combinations thereof). In embodiments, if a symptom of esophageal cancer is detected, a diagnostic test for esophageal cancer is performed. In embodiments, the indication of esophageal cancer is detection of abnormal tissue or a tumor. For example, in some cases, abnormal tissue includes cells having dysplasia or cells having high grade dysplasia. The abnormal tissue or tumor, in some cases, is detected by a diagnostic test as described above.

### Methods of Treatment

Provided herein are methods for treating a subject for esophageal cancer, including detecting the presence of extrachromosomal DNA (ecDNA) in a plurality of esophageal cells from a biological sample obtained from the subject. Applicant has discovered that the presence of ecDNA is correlative to the presence of esophageal cancer or precancerous cells that will develop into esophageal cancer. Thus, detecting the presence of ecDNA presents an effective method to treat esophageal cancer patients, particularly at an early stage of the cancer. Moreover, in some cases, detecting the presence of ecDNA prior to administration of an esophageal cancer treatment presents an effective method to detect esophageal cancer at an early stage when other methods known in the art (e.g. Barium swallow test, endoscopy, etc.) are not sensitive enough to detect small numbers of cancerous cells. Thus, in an aspect is provided a method of treating esophageal cancer in a subject in need thereof, the method including: i) obtaining a biological sample containing a plurality esophageal cells from the subject; ii) detecting the presence of extrachromosomal DNA (ecDNA) in the plurality of esophageal cells; and iii) administering an esophageal cancer treatment to the subject.

In instances, a subject who has Barrett's esophagus has cancerous or precancerous cells (e.g. abnormal cells, cells having dysplasia). A subject who has Barrett's esophagus, in instances, has precancerous cells that can develop into esophageal adenocarcinoma (EAC). Thus, in embodiments, the subject has Barrett's esophagus.

For the methods provided herein, in embodiments, the biological sample obtained from the subject includes esophageal epithelial tissue which can be characterized by multilayered squamous epithelial cells. In embodiments, the biological sample includes esophageal epithelium tissue. In subjects who have esophageal cancer or who are at risk of epithelial cancer, the squamous epithelial cells of the esophagus can be replaced by cells not typically found in esophageal tissue. Thus, in embodiments, the plurality of esophageal cells have metaplasia or dysplasia. In embodiments, the plurality of esophageal cells have high grade dysplasia. In embodiments, the plurality of esophageal cells includes columnar epithelial cells. In embodiments, the plurality of esophageal cells includes chief cells, parietal cells, enterocytes, Paneth cells, enteroendocrine cells, or a combination thereof. In embodiments, the plurality of esophageal cells includes characteristics of chief cells, parietal cells, enterocytes, Paneth cells, enteroendocrine cells, or a combination thereof.

Applicant has further demonstrated that the ecDNA count per cell increases during the progression from non-cancerous esophageal epithelial tissue to esophageal cancer (e.g. esophageal adenocarcinoma). Thus, in embodiments, the methods further include determining the ecDNA count per esophageal cell. In embodiments, the method further includes detecting the presence of an amplified oncogene in said plurality of esophageal cells. In embodiments, the method further includes comprising detecting the presence of an oncogene on said ecDNA. In embodiments, the oncogene is independently EGFR (ERBB1), MYC, MYCN, CCND1, ERBB2, CDK4, CDK6, BRAF, MDM2, MDM4, CDC6, CSF3, HMGA1, RARA, THRA, KRAS, CTNNB1, GATA6, TNS4 or combinations thereof.

In embodiments, the method includes administering to said subject an esophageal cancer-preventative treatment prior to administering an esophageal cancer treatment to said subject. In embodiments, the esophageal cancer-preventative treatment is radiofrequency ablation.

In embodiments, the method further includes performing a diagnostic test for esophageal cancer. In embodiments, the method further includes performing a diagnostic test for esophageal cancer prior to administering an esophageal cancer treatment to said subject. In embodiments, the diagnostic test is a barium swallow test, computed tomography (CT) scan, CT-guided needle biopsy, magnetic resonance imaging (MRI) scan, positron emission tomography (PET) scan, endoscopy, endoscopy ultrasound, bronchoscopy, thoracoscopy and laparoscopy, or biopsy.

In embodiments, the treating includes mucosal resection, esophagectomy, radiation therapy, chemotherapy, chemoradiation therapy, laser therapy, electrocoagulation, immunotherapy, or targeted therapy. In embodiments, treating includes administering chemotherapy. In embodiments, the chemotherapy is carboplatin and paclitaxel (Taxol), oxaliplatin and either 5-FU or capecitabine, cisplatin and either 5-fluorouracil (5-FU) or capecitabine, cisplatin and irinotecan (Camptosar), or paclitaxel (Taxol) and either 5-FU or capecitabine. In embodiments, treating includes administering chemotherapy. In embodiments, the chemotherapy is carboplatin and paclitaxel (Taxol). In embodiments, the chemotherapy is oxaliplatin and 5-FU. In embodiments, the chemotherapy is oxaliplatin and capecitabine. In embodiments, the chemotherapy is cisplatin and 5-fluorouracil (5-FU). In embodiments, the chemotherapy is cisplatin and capecitabine. In embodiments, the chemotherapy is cisplatin and irinotecan (Camptosar). In embodiments, the chemotherapy is paclitaxel (Taxol) and 5-FU. In embodiments, the chemotherapy is paclitaxel (Taxol) and capecitabine. In embodiments, treating includes administering targeted therapy. In embodiments, the targeted therapy is cetuximab, bevacizumab, trastuzumab, or combinations thereof. In embodiments, the targeted therapy is cetuximab. In embodiments, the targeted therapy is bevacizumab. In embodiments, the targeted therapy is trastuzumab. In embodiments, treating includes administering immunotherapy. In some cases, the immunotherapy includes checkpoint inhibitors. In some cases, the immunotherapy includes pembrolizumab or nivolumab. In embodiments, the immunotherapy is pembrolizumab. In embodiments, the immunotherapy is nivolumab.

In another aspect is provided a method of treating esophageal cancer in a subject in need thereof, the method including administering an esophageal cancer treatment to the subject wherein the presence of extrachromosomal DNA (ecDNA) in a plurality of esophageal cells obtained from a biological sample from the subject has been detected, and wherein the esophageal cancer treatment is mucosal resection, esophagectomy, radiation therapy, chemotherapy, chemoradiation therapy, laser therapy, electrocoagulation, immunotherapy, or targeted therapy. In embodiments, the esophageal cancer treatment is mucosal resection. In embodiments, the esophageal cancer treatment is esophagectomy. In embodiments, esophageal cancer treatment is radiation therapy. In embodiments, the esophageal cancer treatment is chemotherapy. In embodiments, the esophageal cancer treatment is chemoradiation therapy. In embodiments, the esophageal cancer treatment is laser therapy. In embodiments, the esophageal cancer treatment is electrocoagulation. In embodiments, the esophageal cancer treatment is immunotherapy. In embodiments, the esophageal cancer treatment is targeted therapy.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Introduction to Exemplary Experiments

Barrett's esophagus (BE) is a pre-cancerous metaplastic condition characterized by at least partial replacement of the squamous epithelium of esophagus is replaced by columnar epithelial cells. For example, the columnar epithelial cells often resemble those typically found in the colonic epithelium. Development of BE results in an elevated risk of developing esophageal adenocarcinoma (EAC). The majority of patients with BE do not develop EAC. For example, there is less than a 5-10% lifetime risk, or about 1 in 200 cases, of BE transitioning to EAC per year. However, the markedly elevated risk of EAC from BE leads to costly and frequent endoscopic biopsy screening, in addition to negative psychological effects for the patient. If suspicious histological changes are seen after the development of BE, aggressive treatment is typically started.

The results provided herein show that ecDNA is an early clonal driver, rather than a late subclonal event of EAC development. Further provided are methods that for elucidating the evolution of ecDNA over time in subjects. Based on these studies, Applicant has developed methods for pre-cancer detection and risk stratification. Applicant further describes therapeutic opportunities to prevent the transition of pre-cancer to cancer.

### Example 2: Determination of Whether ecDNA is an Early Clonal Driver Event in the Transition from Pre-Cancer to Cancer

Applicant has shown herein that the presence of ecDNA is correlative with instances of BE that will progress to EAC.

The Amplicon Architect software was previously used to analyze whole genome sequencing data (WGS) from tumor samples for detecting ecDNA. The Amplicon Architext software is described in detail in U.S. Patent Publication No. 2018/0355416A1, which is incorporated herein by reference in its entirety and for all purposes. Applicant applied this analysis to a well-curated longitudinal study of subjects with BE, as described herein.

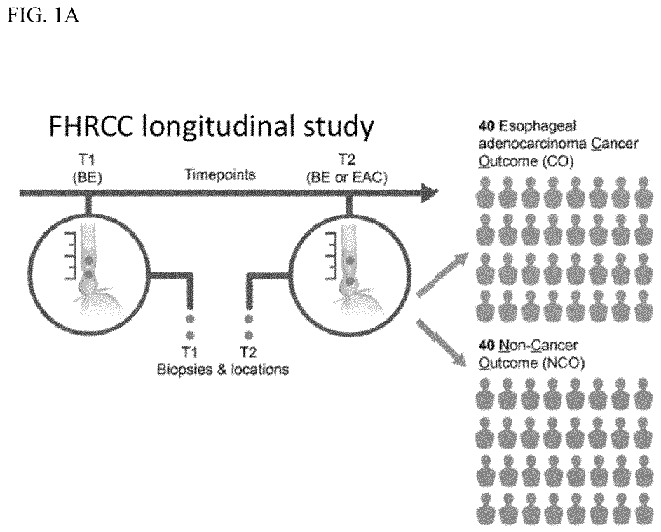

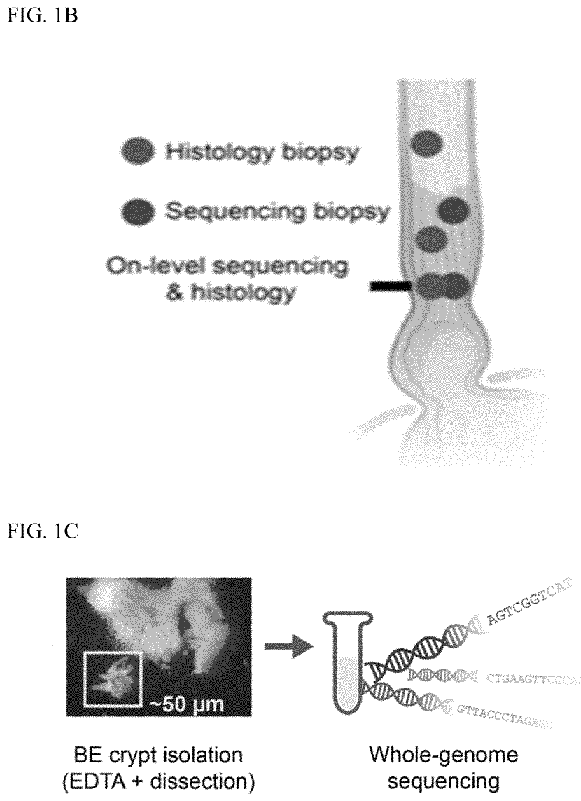

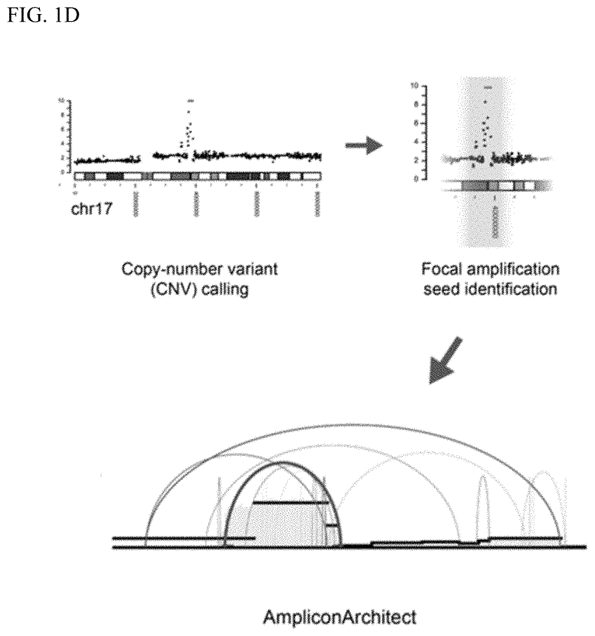

Briefly, the longitudinal study was conducted by the Fred Hutchinson Cancer Research Center (FHCRC) and included an observational study of subjects with BE (FIGS. 1A-1D). The study included 40 subjects whose BE went on to a diagnosis of cancer (EAC) (cancer outcome—CO) and 40 subjects whose BE did not progress to cancer (no cancer outcome—NCO) (FIG. 1A). The subjects underwent two serial surveillance endoscopic examinations from the time of initial diagnosis. Biopsies were taken from multiple regions of the affected area of the esophagus over different time points. A method was further developed to remove the surface epithelial cells from the rest of the esophagus biopsy tissue. The method is described in detail in Li et al. Temporal and spatial evolution of somatic chromosomal alterations: a case-cohort study of Barrett's esophagus. Cancer Prev Res. 2014 January; 7(1):114-27. doi: 10.1158/1940-6207., which is incorporated herein by reference in its entirety and for all purposes. Surface epithelial cells sometimes become abnormal during the development of BE and often progress to EAC. For the biopsies that were completed, attempts were made to confirm, whenever possible, the histology of the region from which the DNA was taken (FIG. 1B). The DNA was then extracted from the cells, followed by whole genome sequencing of the DNA (FIG. 1C).

Applicant analyzed the whole genome sequencing results obtained by FHCRC and used the Amplicon Architect software to detect ecDNA encoded gene amplifications and describe their copy number, contents, and sequence to establish the structure of the ecDNA (FIG. 1D).

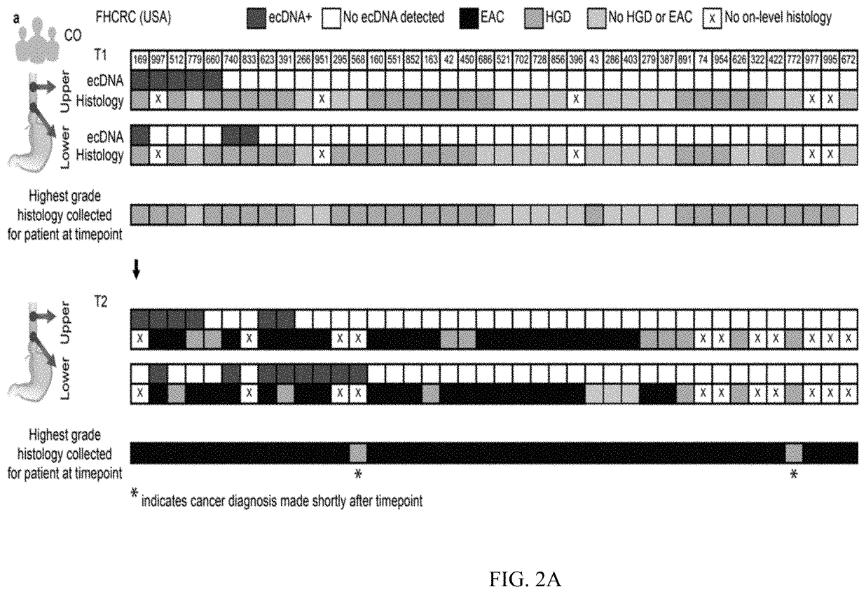

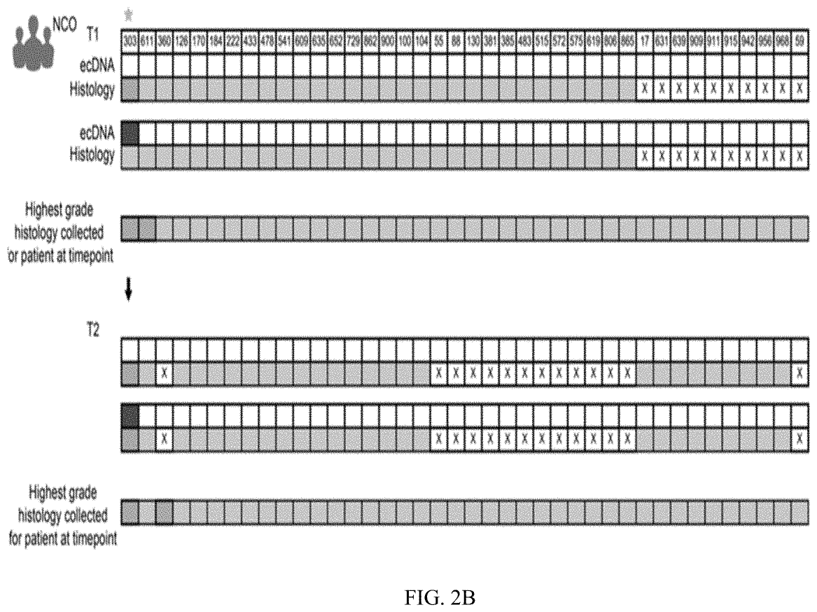

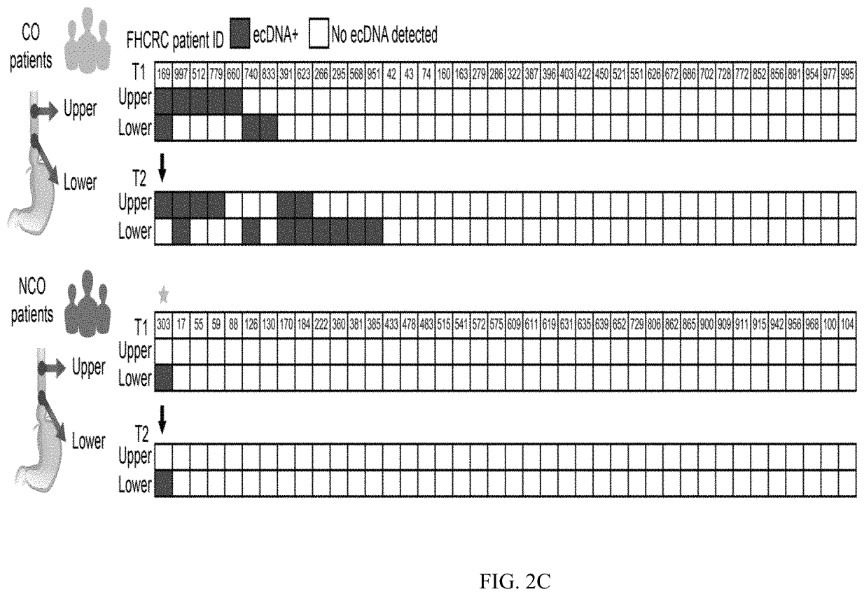

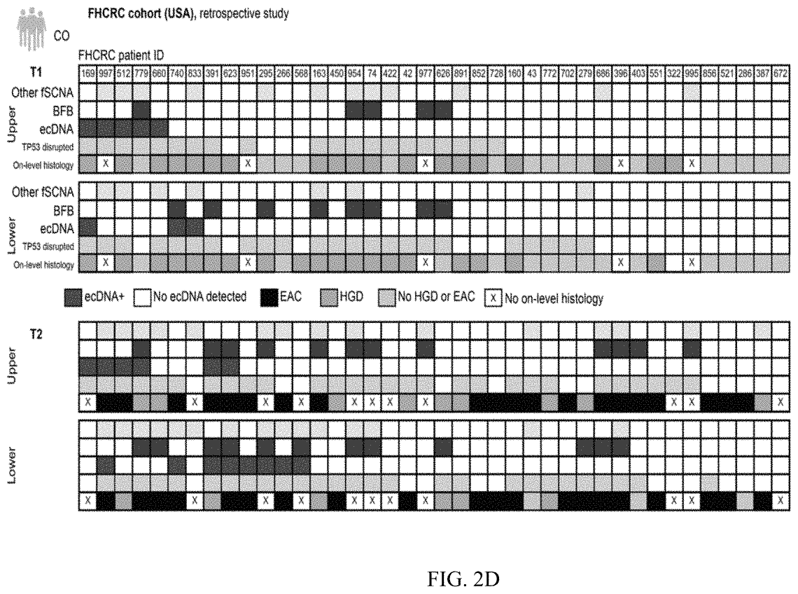

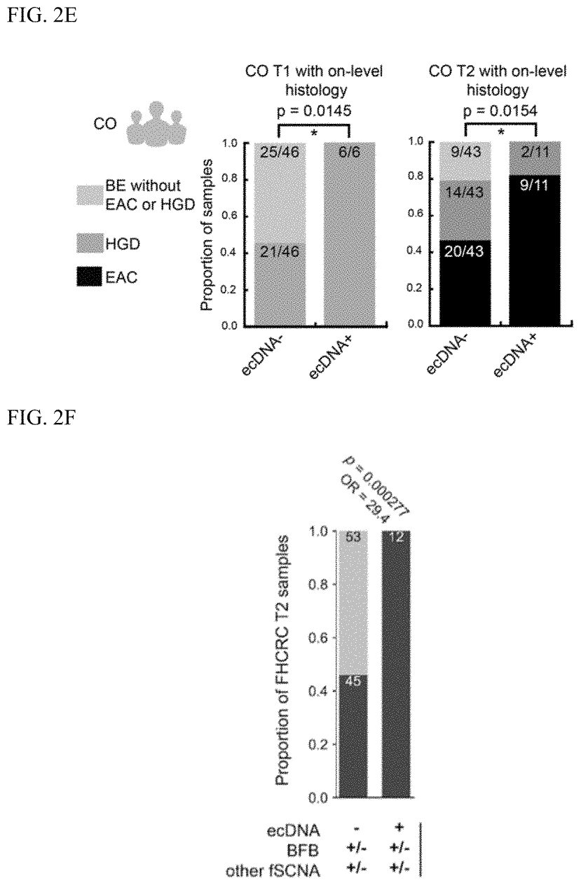

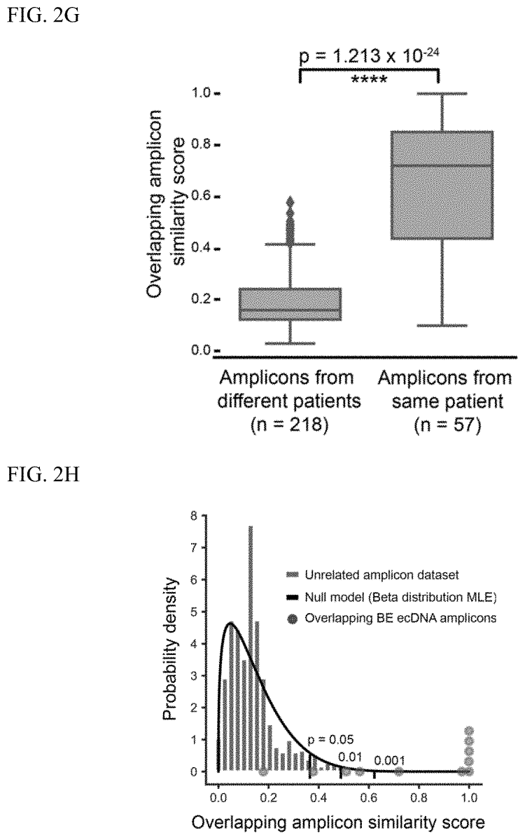

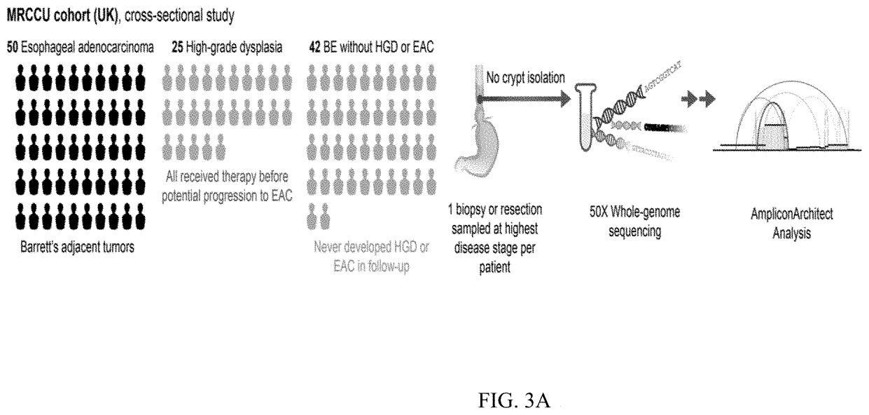

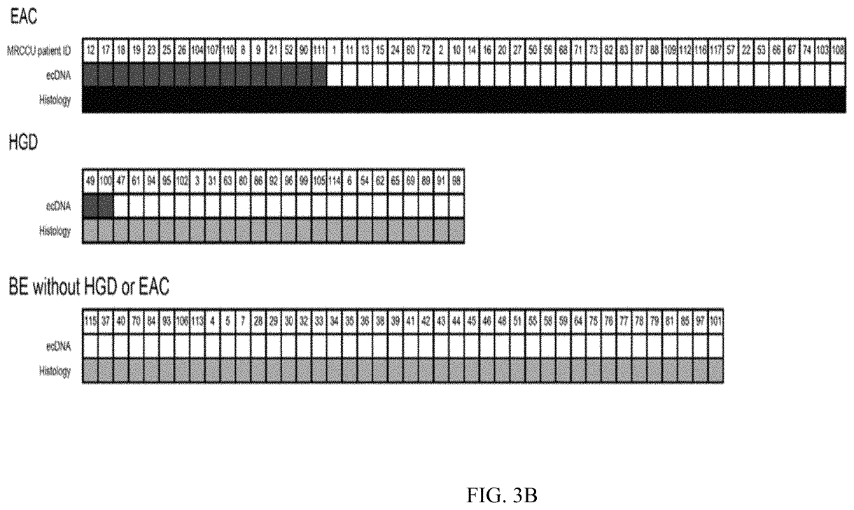

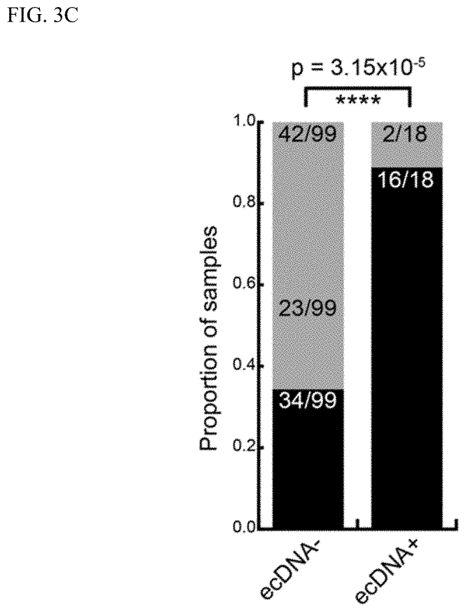

Results from Applicant's analysis reveal how the presence of ecDNA, relative to other types of genetic alterations, is strongly associated with the progression from BE to EAC, occurring in nearly a third of the patients who progressed to EAC (FIGS. 2A-2D). Applicant found that 13 out of 40 subjects who went on to develop EAC from BE had ecDNA in the biological sample from the subject (FIGS. 2A and 2C). In contrast, the only case where ecDNA was present and that didn't progress to cancer (FIG. 2B) was in a single patient who died rapidly from other causes. This patient was suspected of being on the way to developing cancer, with a markedly elevated relative risk of developing EAC from BE. Even for other types of genetic alterations, including oncogene amplification by breakage fusion bridge (BFB) formation, which can co-occur with ecDNA formation, Applicant found ecDNA is even more strongly predictive of developing cancer. Further, detection of the presence of ecDNA was also linked to high grade dysplasia (HGD) on the way to cancer progression in the 11 subjects on whom histology and sequencing were available on the same level of the esophagus (FIG. 2E). Applicant further developed new metrics to determine whether DNA sequences, including oncogenes found on ecDNA on the initial biopsy, are selected for during the progression to cancer. Results illustrate a strong selection for the same regions of ecDNA, indicating that the sequences are selected for during the transformation of BE to EAC (FIGS. 2G and 2H).

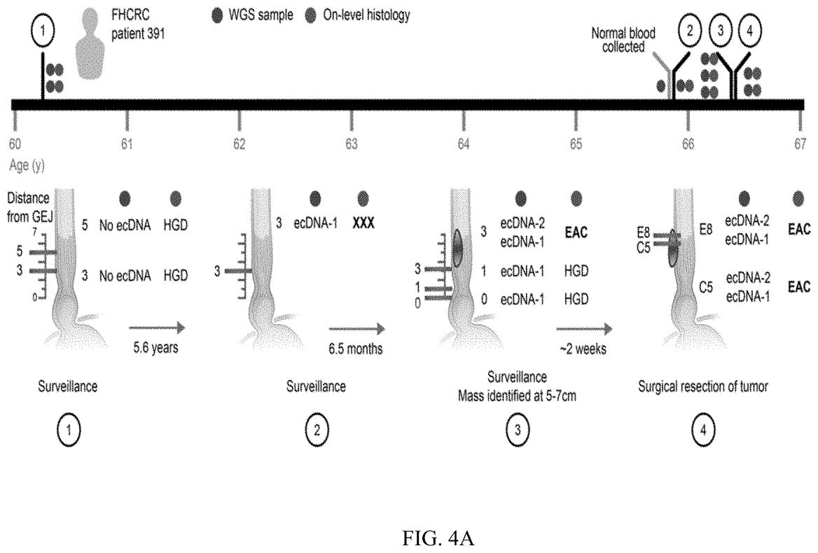

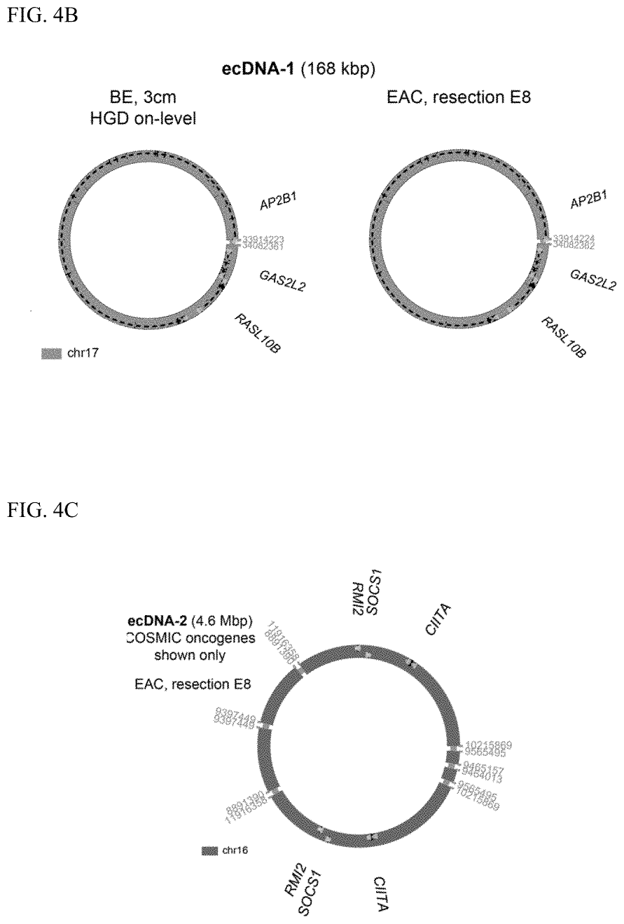

A subject who was enrolled in the early days of the observational study and did not receive any therapy was also studied. This case study thus enabled a true natural history study of cancer evolution without any intervention. Analysis revealed the development and evolution of ecDNA as the subject developed cancer and shows how multiple different ecDNA developed in the BE tissue over time, including ecDNA that contained the oncogenes that were selected and which likely played a key role in transforming the tissue into cancer (FIG. 4A). Results further revealed the overall structure of the circular ecDNA amplicons involved in the cancer transformation in this patient (FIGS. 4B and 4C). This natural history of BE to EAC further showed a central role for ecDNA in driving tumorigenesis.

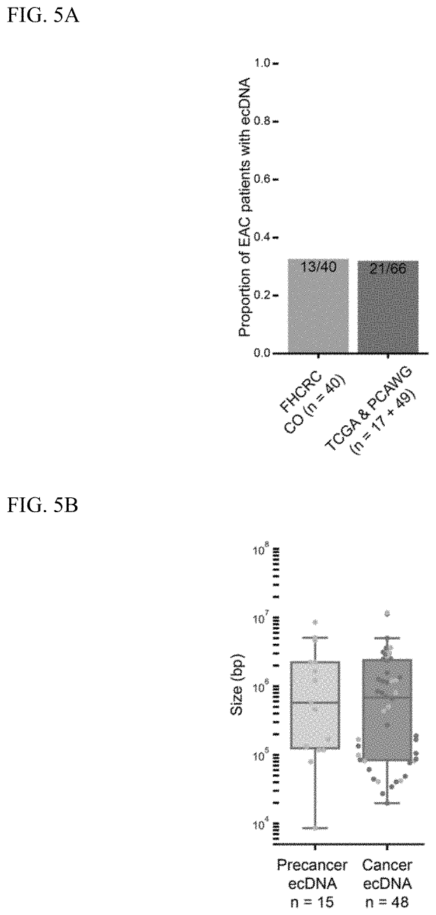

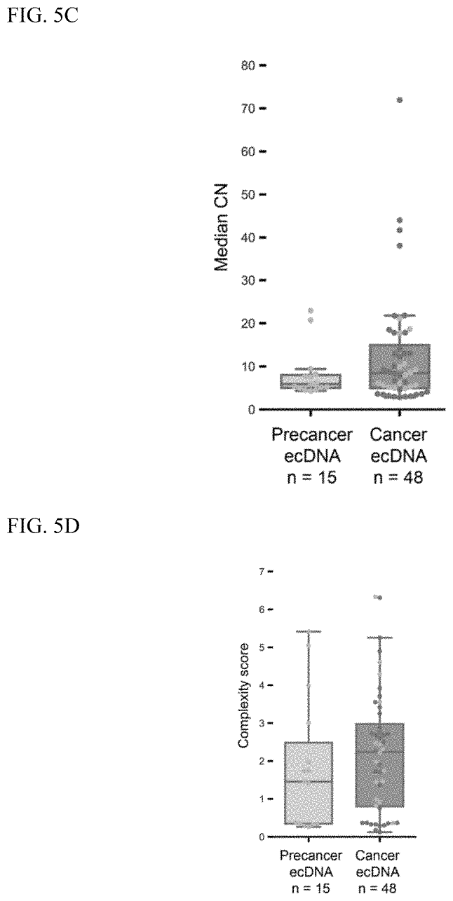

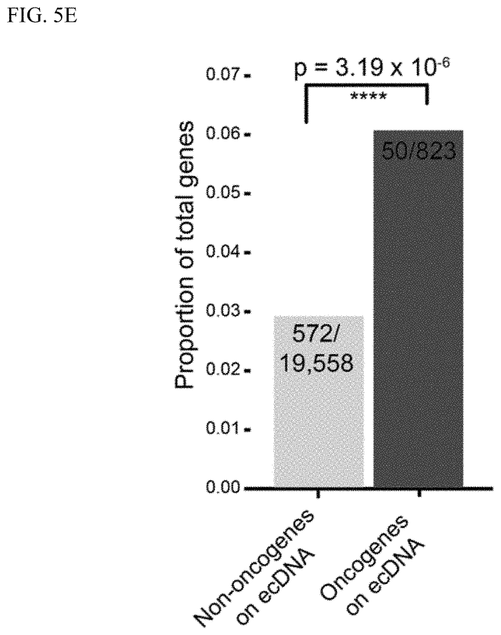

Applicant's sequencing analysis illustrate that the frequency of ecDNA found in the BE subjects that progressed to EAC (32.5%), was almost identical to the fraction of cases of EAC that were found to have ecDNA (31.8%) by Amplicon Architect analysis of the publicly available TCGA and PCAWG datasets (FIG. 5A). These data indicate that the natural frequency of ecDNA in EAC is about one third of patients. Moreover, results show that the general size of the ecDNA amplicons as they progress from initial finding in BE to transformation to cancer do not change significantly (FIG. 5B). However, the copy number of the ecDNA increases, indicating that the ecDNA are being selected for during the transformation to cancer (FIG. 5C). Applicants further developed a complexity score to determine whether the circles themselves are evolving in structure over time, and results show that the circles are changing (FIG. 5D). Taken together with the increased copy number over time, and the increased evolution over time during progression to cancer, the data provide additional support for the role of these ecDNAs in the evolution of BE to EAC.

Analysis further shows that the oncogenes are massively enriched on ecDNA relative to non-ecDNA regions of the genome in BE (FIG. 5E), as illustrated by Applicant's determination of the portion of all of the genes in a defined region of DNA that are oncogenes. Collectively, the results show that the ecDNA that are initially detected in BE contain oncogenes and become selected for, and further evolve, as the tissue transforms into EAC.

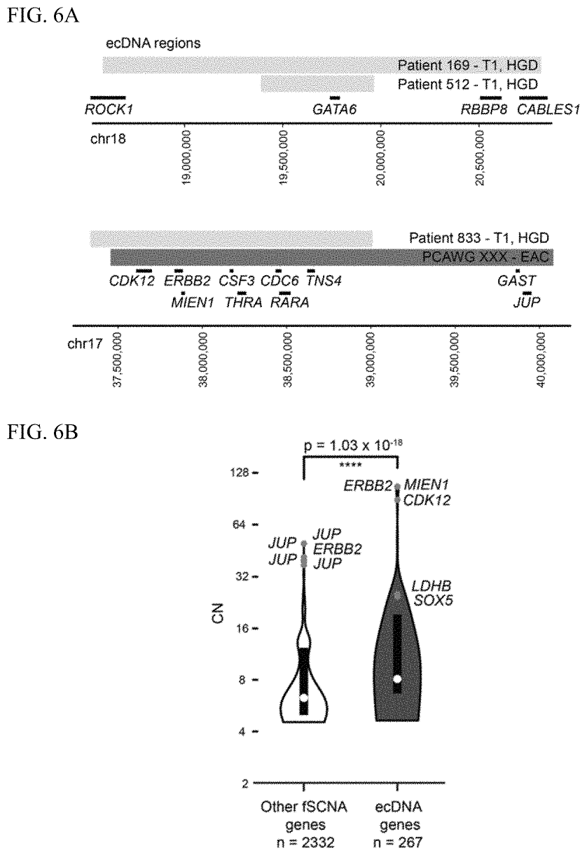

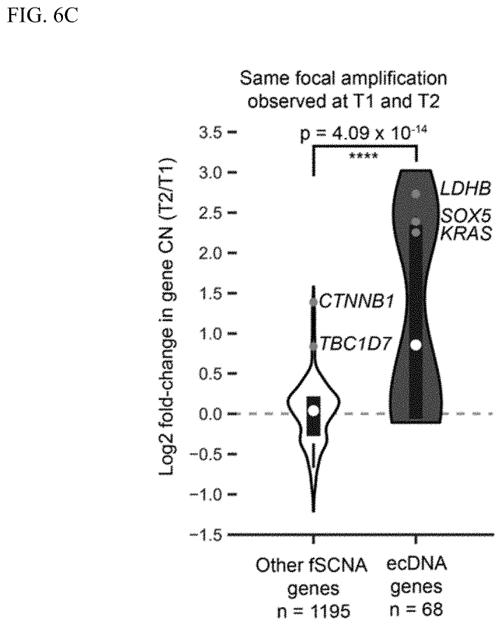

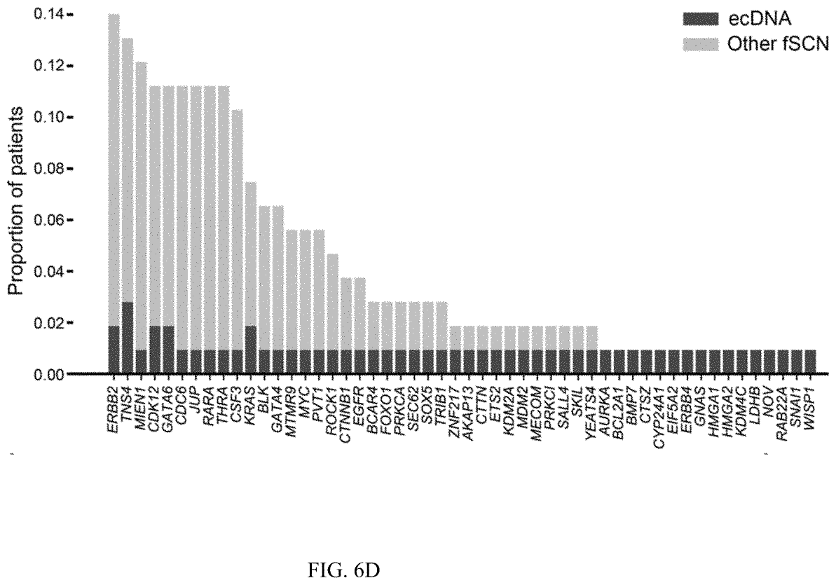

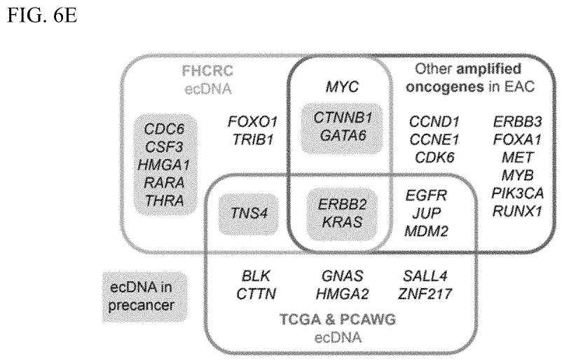

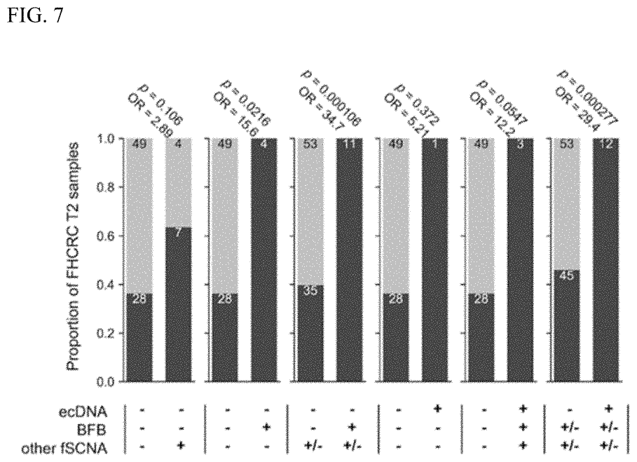

In addition to enrichment of oncogenes in ecDNA, comparative analysis shows that the oncogenes have a higher copy number when amplified on ecDNA than when amplified on chromosomal DNA (FIG. 6B). Further, specific oncogenes are selected for as shown by comparison of copy numbers of the oncogenes between T1 and T2 (FIG. 6C). This analysis indicates that the increase in copy number is linked to the transformation of the Barrett's tissue into cancer. Additionally, Applicant's results show that ecDNA contain a large array of different oncogenes, including a variety of potent oncogenes that drive tumor development (FIG. 6D). Comparison between data sets show a strong convergence between oncogenes found in ecDNA in the BE to EAC transition in the FHCRC data set, oncogenes amplified on ecDNA in the EAC from the TCGA and PCAWG data sets, and other oncogenes amplified in EAC from the TCGA and PCAWG data sets that were not found specifically on ecDNA (FIG. 6E). This data indicates that oncogenes most commonly associated with EAC are frequently found amplified on ecDNA in BE that progresses to EAC. Finally, analysis of the odds ratios of various types of genetic alterations, demonstrates that ecDNA amplification is associated with a nearly 30-fold increase in the risk for developing EAC (FIG. 7).

Consequently, ecDNA can be an early event driving the transition from a pre-cancer state to a cancerous state. Results described herein show that ecDNA is a primary driver of cancer development, not just a late consequence of genome instability. Moreover, ecDNA in BE biopsies strongly predicted cancer development, providing a powerful biomarker for risk stratification and early intervention in subjects with BE. The identification of a subject at risk for progression of BE to EAC provides the opportunity for subject stratification, such as for indicating which patients should be guided towards preventative treatment (e.g., in advance of such progression) and the provision of cancer treatments for EAC. The stratification of subjects at lower risk or not at risk for progression of BE to EAC permits the adoption of a watch and wait course of treatment.

What is claimed is:

1. A method for detecting extrachromosomal DNA (ecDNA) in a subject who has esophageal cancer or is at risk of developing esophageal cancer; said method comprising:
i) obtaining a biological sample containing a plurality esophageal cells from said subject; and
ii) detecting the presence of an extrachromosomal DNA (ecDNA) in said plurality of esophageal cells.

2. The method of claim 1, wherein said subject has Barrett's esophagus.

3. The method of claim 1 or 2, wherein said biological sample comprises esophageal epithelium tissue.

4. The method of any one of claims 1-3, wherein said plurality of esophageal cells have metaplasia or dysplasia.

5. The method of any one of claims 1-4, wherein said plurality of esophageal cells have high grade dysplasia.

6. The method of any one of claims 1-5, wherein said plurality of esophageal cells comprise columnar epithelial cells.

7. The method of any one of claims 1-6, wherein said plurality of esophageal cells comprise chief cells, parietal cells, enterocytes, Paneth cells, enteroendocrine cells, or a combination thereof.

8. The method of any one of claims 1-7, further comprising determining the ecDNA count per esophageal cell in said plurality of esophageal cells.

9. The method of any one of claims 1-8, further comprising detecting the presence of an amplified oncogene in said plurality of esophageal cells.

10. The method of any one of claims 1-9, further comprising detecting the presence of the oncogene on said ecDNA.

11. The method of claim 9 or 10, wherein said oncogene is independently EGFR (ERBB1), MYC, MYCN, CCND1, ERBB2, CDK4, CDK6, BRAF, MDM2, MDM4, CDC6, CSF3, HMGA1, RARA, THRA, KRAS, CTNNB1, GATA6, TNS4 or a combination thereof.

12. The method of any one of claims 1-11, wherein the subject is at an elevated risk of esophageal cancer if the presence of ecDNA is detected in the plurality of esophageal cells, relative to a subject wherein the presence of ecDNA is not detected in the plurality of esophageal cells.

13. The method of any one of claims 1-12, comprising administering to said subject an esophageal cancer-preventative treatment.

14. The method of claim 13, wherein said esophageal cancer-preventative treatment is radiofrequency ablation.

15. The method of any one of claims 1 to 14, further comprising performing a diagnostic test for esophageal cancer on said subject.

16. The method of claim 15, wherein said diagnostic test is barium swallow test, computed tomography (CT) scan, CT-guided needle biopsy, magnetic resonance imaging (MRI) scan, positron emission tomography (PET) scan, endoscopy, endoscopy ultrasound, bronchoscopy, thoracoscopy and laparoscopy, or biopsy.

17. The method of any of claims any one of claims 1-16, further comprising treating said subject for esophageal cancer.

18. The method of claim 17, wherein said treating comprises mucosal resection, esophagectomy, radiation therapy, chemotherapy, chemoradiation therapy, laser therapy, electrocoagulation, immunotherapy, or targeted therapy.

19. The method of any of claims any one of claims 1-7, further comprising placing the subject on a watch and wait course, wherein ecDNA is not detected the said plurality of esophageal cells.

20. A method of treating esophageal cancer in a subject in need thereof, said method comprising:
i) obtaining a biological sample containing a plurality esophageal cells from said subject;
ii) detecting the presence of extrachromosomal DNA (ecDNA) in said plurality of esophageal cells; and
iii) administering an esophageal cancer treatment to said subject.

21. The method of claim 20, wherein said subject has Barrett's esophagus.

22. The method of claim 20 or 21, wherein said biological sample comprises esophageal epithelium tissue.

23. The method of any one of claims 20-22, wherein said plurality of esophageal cells have metaplasia or dysplasia.

24. The method of any one of claims 20-23, wherein said plurality of esophageal cells have high grade dysplasia.

25. The method of any one of claims 20-24, wherein said plurality of esophageal cells comprise columnar epithelial cells.

26. The method of any one of claims 20-25, wherein said plurality of esophageal cells comprise chief cells, parietal cells, enterocytes, Paneth cells, enteroendocrine cells, or a combination thereof.

27. The method of any one of claims 20-26, further comprising determining the ecDNA count per esophageal cell.

28. The method of any one of claims 20-27, further comprising detecting the presence of an amplified oncogene in said plurality of esophageal cells.

29. The method of any one of claims 20-28, further comprising detecting the presence of an oncogene on said ecDNA.

30. The method of claim 28 or 29, wherein said oncogene is independently EGFR (ERBB1), MYC, MYCN, CCND1, ERBB2, CDK4, CDK6, BRAF, MDM2, MDM4, CDC6, CSF3, HMGA1, RARA, THRA, KRAS, CTNNB1, GATA6, TNS4 or combinations thereof.

31. The method of any one of claims 20-30, comprising administering to said subject an esophageal cancer-preventative treatment prior to administering an esophageal cancer treatment to said subject.

32. The method of claim 31, wherein said esophageal cancer-preventative treatment is radiofrequency ablation.

33. The method of any one of claims 20-32, further comprising performing a diagnostic test for esophageal cancer prior to administering an esophageal cancer treatment to said subject.

34. The method of claim 33, wherein said diagnostic test is a barium swallow test, computed tomography (CT) scan, CT-guided needle biopsy, magnetic resonance imaging (MRI) scan, positron emission tomography (PET) scan, endoscopy, endoscopy ultrasound, bronchoscopy, thoracoscopy and laparoscopy, or biopsy.

35. The method of any one of claims 20-34, wherein said treating comprises mucosal resection, esophagectomy, radiation therapy, chemotherapy, chemoradiation therapy, laser therapy, electrocoagulation, immunotherapy, or targeted therapy.
